SEQUENCE LISTING (53 sequences)

SEQ ID NO: 1 - protein, 196 aa, Unknown
  Description of Unknown FokI cleavage half domain sequence
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 2 - protein, 7 aa, Artificial Sequence
  Description of Artificial Sequence Synthetic peptide
LPHHLEQ

SEQ ID NO: 3 - protein, 7 aa, Artificial Sequence
  Description of Artificial Sequence Synthetic peptide
RNASRTR

SEQ ID NO: 4 - protein, 7 aa, Artificial Sequence
  Description of Artificial Sequence Synthetic peptide
YTYSLSE

SEQ ID NO: 5 - protein, 7 aa, Artificial Sequence
  Description of Artificial Sequence Synthetic peptide
QSANRTT

SEQ ID NO: 6 - protein, 7 aa, Artificial Sequence
  Description of Artificial Sequence Synthetic peptide
HRSSLRR

SEQ ID NO: 7 - protein, 7 aa, Artificial Sequence
  Description of Artificial Sequence Synthetic peptide
GNTGLNC

SEQ ID NO: 8 - protein, 7 aa, Artificial Sequence
  Description of Artificial Sequence Synthetic peptide
DRSNLTR

SEQ ID NO: 9 - protein, 7 aa, Artificial Sequence
  Description of Artificial Sequence Synthetic peptide
QSGSLTR

SEQ ID NO: 10 - protein, 7 aa, Artificial Sequence
  Description of Artificial Sequence Synthetic peptide
HKSARAA

SEQ ID NO: 11 - protein, 7 aa, Artificial Sequence
  Description of Artificial Sequence Synthetic peptide
RSDHLSA

SEQ ID NO: 12 - protein, 7 aa, Artificial Sequence
  Description of Artificial Sequence Synthetic peptide
QHGALQT

SEQ ID NO: 13 - protein, 7 aa, Artificial Sequence
  Description of Artificial Sequence Synthetic peptide
RSDHLSQ

SEQ ID NO: 14 - protein, 7 aa, Artificial Sequence
  Description of Artificial Sequence Synthetic peptide
QSNGLTQ

SEQ ID NO: 15 - protein, 7 aa, Artificial Sequence
  Description of Artificial Sequence Synthetic peptide
QHGSLAS

SEQ ID NO: 16 - protein, 7 aa, Artificial Sequence
  Description of Artificial Sequence Synthetic peptide
RSAHLSA

SEQ ID NO: 17 - protein, 7 aa, Artificial Sequence
  Description of Artificial Sequence Synthetic peptide
SSSHRCQ

SEQ ID NO: 18 - protein, 7 aa, Artificial Sequence
  Description of Artificial Sequence Synthetic peptide
QRVALQA

SEQ ID NO: 19 - protein, 7 aa, Artificial Sequence
  Description of Artificial Sequence Synthetic peptide
QSGALAR

SEQ ID NO: 20 - protein, 7 aa, Artificial Sequence
  Description of Artificial Sequence Synthetic peptide
YRWLRNS

SEQ ID NO: 21 - protein, 7 aa, Artificial Sequence
  Description of Artificial Sequence Synthetic peptide
WYTARYQ

SEQ ID NO: 22 - protein, 7 aa, Artificial Sequence
  Description of Artificial Sequence Synthetic peptide
QRTHLTQ

SEQ ID NO: 23 - protein, 7 aa, Artificial Sequence
  Description of Artificial Sequence Synthetic peptide
DRSNLSR

SEQ ID NO: 24 - protein, 7 aa, Artificial Sequence
  Description of Artificial Sequence Synthetic peptide
QQANRKK

SEQ ID NO: 25 - protein, 7 aa, Artificial Sequence
  Description of Artificial Sequence Synthetic peptide
RPYTLRL

SEQ ID NO: 26 - protein, 7 aa, Artificial Sequence
  Description of Artificial Sequence Synthetic peptide
QSGHLAR

SEQ ID NO: 27 - protein, 7 aa, Artificial Sequence
  Description of Artificial Sequence Synthetic peptide
QSSNRQK

SEQ ID NO: 28 - protein, 7 aa, Artificial Sequence
  Description of Artificial Sequence Synthetic peptide
RSFSLQV

SEQ ID NO: 29 - protein, 7 aa, Artificial Sequence
  Description of Artificial Sequence Synthetic peptide
RTYSLAV

SEQ ID NO: 30 - protein, 7 aa, Artificial Sequence
  Description of Artificial Sequence Synthetic peptide
RNFSLTM

SEQ ID NO: 31 - protein, 7 aa, Artificial Sequence
  Description of Artificial Sequence Synthetic peptide
QWYGRSN

SEQ ID NO: 32 - protein, 7 aa, Artificial Sequence
  Description of Artificial Sequence Synthetic peptide
QSANRTK

SEQ ID NO: 33 - DNA, 28 bp, Artificial Sequence
  Description of Artificial Sequence Synthetic oligonucleotide
aagttaaacttgtgtgttttcttagggc

SEQ ID NO: 34 - DNA, 28 bp, Artificial Sequence
  Description of Artificial Sequence Synthetic oligonucleotide
tgataaggattgtaagacttcatcctac

SEQ ID NO: 35 - DNA, 28 bp, Artificial Sequence
  Description of Artificial Sequence Synthetic oligonucleotide
gataaggattgtaagacttcatcctaca

SEQ ID NO: 36 - DNA, 24 bp, Artificial Sequence
  Description of Artificial Sequence Synthetic primer
atactagtccgcgagccttcaaag

SEQ ID NO: 37 - DNA, 24 bp, Artificial Sequence
  Description of Artificial Sequence Synthetic primer
gagggttccgatatctttgtgatt

SEQ ID NO: 38 - DNA, 20 bp, Artificial Sequence
  Description of Artificial Sequence Synthetic primer
aagtttaacttctgataagg

SEQ ID NO: 39 - DNA, 20 bp, Artificial Sequence
  Description of Artificial Sequence Synthetic primer
tgctagccgcaggcattact

SEQ ID NO: 40 - DNA, 23 bp, Artificial Sequence
  Description of Artificial Sequence Synthetic primer
cgggatcaaagaaagttgtgttt

SEQ ID NO: 41 - DNA, 24 bp, Artificial Sequence
  Description of Artificial Sequence Synthetic primer
gcctacactggagtccgtgctact

SEQ ID NO: 42 - DNA, 24 bp, Artificial Sequence
  Description of Artificial Sequence Synthetic primer
ctgaccacacacgagctggtagaa

SEQ ID NO: 43 - DNA, 45 bp, Artificial Sequence
  Description of Artificial Sequence Synthetic oligonucleotide
taatacgactcactatagggagactaaggactgtaagacttcatc

SEQ ID NO: 44 - DNA, 21 bp, Artificial Sequence
  Description of Artificial Sequence Synthetic oligonucleotide
gaggtcttagcttaattaaag

SEQ ID NO: 45 - DNA, 45 bp, Artificial Sequence
  Description of Artificial Sequence Synthetic oligonucleotide
taatacgactcactatagggagacaagtcttagtagagatttctc

SEQ ID NO: 46 - DNA, 21 bp, Artificial Sequence
  Description of Artificial Sequence Synthetic oligonucleotide
ggtcttaaggtgatattcatg

SEQ ID NO: 47 - DNA, 52 bp, Artificial Sequence
  Description of Artificial Sequence Synthetic oligonucleotide
aagcctacagcacccggtattcccaggcggtctcccatccaagtactaacca

SEQ ID NO: 48 - protein, 7 aa, Artificial Sequence
  Description of Artificial Sequence Synthetic peptide
RSFTLMQ

SEQ ID NO: 49 - protein, 9 aa, Unknown
  Description of Unknown “LAGLIDADG” family peptide motif sequence
LAGLIDADG

SEQ ID NO: 50 - DNA, 47 bp, Unknown
  Description of Unknown wildtype mtDNA oligonucleotide
gaaaacacacaagtttaacttctgataaggactgtaagacttcatcc

SEQ ID NO: 51 - DNA, 47 bp, Artificial Sequence
  Description of Artificial Sequence Synthetic oligonucleotide
gaaaacacacaagtttaacttctgataaggattgtaagacttcatcc

SEQ ID NO: 52 - DNA, 43 bp, Mus sp.
aaacacacaagtttaacttctgataaggattgtaagacttcat

SEQ ID NO: 53 - RNA, 69 nt, Artificial Sequence
  Description of Artificial Sequence Synthetic oligonucleotide
gaggucuuagcuuaauuaaagcaauugauuugcauucaauagauguaggaugaagucuuacaauccuua

(19) United States
(12) Patent Application Publication
(10) Pub. No.: US 2021/0002670 A1
(43) Pub. Date: Jan. 7, 2021

(54) GENETIC MODIFICATION OF MITOCHONDRIAL GENOMES

(71) Applicants: Michal Minczuk, Cambridge (GB); Payam A. Gammage, Glasgow (GB); Sangamo Therapeutics, Inc., Brisbane, CA (US); The Chancellor, Masters and Scholars of The University of Cambridge, Cambridge (GB)

(72) Inventors: Michal Minczuk, Cambridge (GB); Lei Zhang, Brisbane, CA (US); Payam A. Gammage, Glasgow (GB)

(21) Appl. No.: 16/982,941

(22) PCT Filed: Mar. 21, 2019
(86) PCT No.: PCT/US2019/023359

§ 371 Date: Sep. 21, 2020

Related U.S. Application Data
(60) Provisional application No. 62/646,156, filed on Mar. 21, 2018.

(51) Int. Cl. C12N 15/90; C12N 15/63; C12N 9/22

(52) U.S. Cl. CPC C12N 15/907; C12N 2750/14143; C12N 9/22; C12N 15/63

(57) ABSTRACT

The present disclosure is in the field of genome engineering, particularly targeted genetic modification of mitochondrial DNA (mtDNA).

### CROSS-REFERENCE TO RELATED APPLICATIONS

The present application claims the benefit of U.S. Provisional Application No. 62/646,156, filed Mar. 21, 2018, the disclosure of which is hereby incorporated by reference in their entirety.

### TECHNICAL FIELD

The present disclosure is in the field of genome engineering, particularly targeted modification of a mitochondrial genome (mtDNA).

### BACKGROUND

Mitochondrial diseases are a genetically diverse group of hereditary, multi-system disorders (often affecting organs requiring the greatest amount of energy such as heart, brain, muscles and lungs), the majority of which are transmitted through mutation of mitochondrial DNA (mtDNA), affecting approximately 1 in 5,000 adults. See, e.g., Gorman et al. (2015) Ann Neurol 77:753-759. There are at least 250 pathogenic mtDNA mutations characterized thus far (Tuppen et al (2010) Biochem Biophys Acta 1797:113-128), and these mutations appear to play a role in several types of human disease. Human mtDNA is a small, double-stranded, multi-copy genome present at ˜100-10,000 copies per cell. In the disease state, mutated mtDNA often co-exists with wild-type mtDNA in a phenomenon known as “heteroplasmy” (resulting from the maternal inheritance of a plurality of mitochondria through the ovum). As mutant mtDNA is typically functionally recessive, the presence of mutated mtDNA is facilitated by wild-type genomes, and disease severity in conditions caused by heteroplasmic mtDNA mutations correlates with mutation load. See, e.g., Gorman et al. (2016) Nat Rev Dis Primers 2:16080. A threshold effect, where >60% mutant mtDNA load must be exceeded before symptoms manifest, is a definitive feature of heteroplasmic mtDNA diseases, and attempts to shift the heteroplasmic ratio below this threshold have driven much research towards treatment of these incurable and essentially untreatable disorders.

One such approach relies on directed nucleolysis of mtDNA using, among other genome engineering tools, mitochondrially targeted zinc finger-nucleases (mtZFNs). See, e.g., Srivastava et al. (2001) Hum Mol Genet 10: 3093-3099 (2001); Bacman et al. (2013) Nat Med 19:1111-1113; Gammage et al. (2014) EMBO Mot Med 6:458-466; Reddy et al. (2015) Cell 161:459-469; Gammage et al. (2016) Nucleic Acids Res 44:7804-7816; Gammage et al (2018) Trends Gene 34(2):101). Because mammalian mitochondria lack efficient DNA double-strand break (DSB) repair pathways, selective introduction of DSBs into mutant mtDNA leads to rapid degradation of these molecules through an incompletely characterized mechanism. As mtDNA copy number is maintained at a cell type-specific steady-state level, selective elimination of mutant mtDNA stimulates replication of the remaining mtDNA pool, eliciting shifts in the heteroplasmic ratio. Methods for delivery of ZFNs to mitochondria in cultured cells has been shown to be capable of producing large heteroplasmic shifts that result in the phenotypic rescue of patient-derived cell cultures. See, e.g., Minczuk et al. (2006) Proc Natl Acad Sci USA 103:19689-19694 (2006); Minczuk, et al. (2010) Nat Protoc 5:342-356; Minczuk et al., (2008) Nucleic Acids Res 36:3926-3938; Gaude et al. (2018) Mol Cell 69:581-593; U.S. Pat. No. 9,139,628.

Despite the initial descriptions of mtDNA mutations associated with human disease emerging in the late 1980's (see, e.g., Holt et al. (1988) Nature 331:717-719; Wallace et al. (1988) Science 242:1427-1430; Wallace et al. (1988) Cell 55:601-610), effective treatments for heteroplasmic mitochondrial disease have not been forthcoming in the intervening decades. Preventing the transmission of mtDNA mutations through mitochondrial replacement therapy has gained traction (see, e.g., Craven et al. (2010) Nature 465:82-85; Tachibana et al. (2013) Nature 493:627-631; Hyslop et al. (2016) Nature 534:383-386; Kang et al. (2016) Nature 540:270-275), although given the nature of the mtDNA bottleneck (Floros et al. (2018) Nat Cell Biol 20:144-151), heterogeneous mitochondrial disease presentation (Vafai et al. (2012) Nature 491:374-383) and subsequent lack of family history of mitochondrial disease in the majority of new cases, mitochondrial replacement can only be of limited use. In addition, molecular pathways for treatment of mitochondrial disease have not provided clinically-relevant therapies for heteroplasmic mitochondrial disease. See, e.g., Viscomi et al. (2015) Biochim Biophy Acta 1847:544-557; Pfeffer et al. (2013) Nat Rev Neurol 9:474-481.

Thus, there remains a need for additional methods and compositions for mtDNA gene modification, particularly heteroplasmy shifting of mtDNA to provide a universal therapeutic for treatment of mitochondrial diseases of diverse genetic origin by reducing the amount of mutant mitochondrial sequences.

### SUMMARY

The present invention describes compositions and methods for use in gene therapy and genome engineering. Specifically, the methods and compositions described relate to nuclease-mediated genomic modification (e.g., one or more insertions and/or deletions) of an endogenous mitochondrial genome (mutant or wild-type). The mitochondrial genome may be altered for targeted correction of a disease-causing mutation, including by nuclease-mediated shifting of the ratio of mutant and wild type mtDNAs in a subject with a mitochondrial disease, including in one or more specific tissues and/or organs (for example in cardiac tissue) that results in phenotypic reversion of the targeted tissues to wild-type (e.g., molecular and biochemical phenotypes). This reversion occurs through heteroplasmy shifting where the ratio of mutant and wild type mtDNAs is altered by cleaving the mutant sequence such that, in the absence of efficient DNA-repair mechanisms (as in mitochondria), the mutant, disease associate mtDNA is degraded after selective cleavage by targeted nucleases.

Thus, the genomic modification(s) (e.g., heteroplasmy shifting) may comprise cleavage followed by degradation of the cleaved mtDNA sequence, and these genetic modifications and/or cells comprising these modifications may be used in ex vivo or in vivo methods.

Thus, described herein is use of (or a pharmaceutical composition comprising) a zinc finger nuclease comprising left and right zinc finger nucleases (ZFNs) for treatment of a mitochondrial disorder in a subject in need thereof, wherein one ZFN partner comprises a cleavage domain and a zinc finger protein (ZFP) that binds to a target site in mutant mitochondrial DNA (mutant mtDNA), and the other ZFN partner comprises a cleavage domain and a zinc finger protein (ZFP) that binds to a target site in either a wild type mitochondrial DNA (mtDNA) or a mutant mtDNA (mutant mtDNA) such that mutant mtDNA in the subject is reduced or eliminated (e.g., shifting the heteroplasmic ratio of wild-type to mutant mtDNA). In some embodiments, both the right and left ZFPs bind to targets in mutant mtDNA, while in other embodiments, one ZFN partner binds to wildtype mtDNA and the other ZFN partner binds to mutant mtDNA. In further embodiments, the ZFN that binds to the wildtype mtDNA is the left ZFN while the right ZFN binds to the mutant mtDNA, or the right ZFN binds to the wildtype mtDNA while the left ZFN binds to the mutant mtDNA. Also described are methods of treating a mitochondrial disorder in a subject in need thereof by expressing the ZFNs described herein in the subject. In any of the uses or methods described herein, the mutant mtDNA comprises one or more of the following mutations: 5024C>T, 1555G, 1624T, 3243G, 3460A, 3271C, 4300G, 5545T, 7445G, 7472 random insertions, 8344G, 8356C 8993G, 9176G/C, 10158C, 10191C, 10197A, 11777A, 11778A, 13513A, 14459A, 14484C, 14487C and/or 14709C. In certain embodiments, the zinc finger nuclease is encoded by one or more polynucleotides (e.g., separate polynucleotides encoding the left and right ZFNs or the same polynucleotide encoding both left and right ZFNs), including but not limited to one or more polynucleotides carried by one or more AAV vectors. The subject may be a human subject and the mtDNA may be in any tissue of the subject. In some embodiments, the mtDNA may be in the brain, lung and/or muscle of the subject. The ZFNs and/or polynucleotides may be administered by any suitable means, including intravenous injection. In embodiments in which the mutant mtDNA comprises the 5024C>T mutation, the left ZFP may bind to a target site within SEQ ID NO:33 and the right ZFP may bind to a target site within SEQ ID NO:34, including but not limited to a ZFN in which the left ZFN comprises a ZFP designated WTM1/48960 and the right ZFN comprises a ZFP designated MTM62/48962, MTM24/51024, MTM25/51025, MTM26/51026, MTM27/51027, MTM28/51028, MTM29/51029, MTM30/51030, MTM32/51032, MTM33/51033, MTM36/51036, MTM37/51037, MTM39/51039, MTM42/51042, MTM43/51043 or MTM45/51045.

Also described is a zinc finger nuclease comprising left and right zinc finger nucleases (ZFNs), wherein the left ZFN comprises a cleavage domain and zinc finger protein (ZFP) that binds to a target site in wild-type mitochondrial DNA within SEQ ID NO:33 and the right ZFN comprises a cleavage domain and a ZFP that binds to a target site in mutant mitochondrial DNA within SEQ ID NO:34 or SEQ ID NO:35. In certain embodiments, the ZFN is encoded by one or more polynucleotides (e.g., carried by AAV vectors). Cells (e.g., cardiac, brain, lung and/or muscle cells) comprising the nucleases and/or polynucleotides as set forth herein are also described, including cells in which mutant mtDNA at position 5024 in the cell is reduced or eliminated as well as cells, cell lines and partially or fully differentiated cells descended from these cells (that may not include the ZFN or polynucleotide encoding the ZFN). Pharmaceutical compositions comprising one or more zinc finger nucleases; one or more polynucleotides and/or the cell as described herein are also provided.

In one aspect, disclosed herein are methods and compositions for targeted modification of mtDNA gene using one or more nucleases. Nucleases, for example engineered meganucleases, zinc finger nucleases (ZFNs) (the term “a ZFN” includes a pair of ZFNs), TALE-nucleases (TALENs including fusions of TALE effectors domains with nuclease domains from restriction endonucleases and/or from meganucleases (such as mega TALEs and compact TALENs) (the term “a TALEN” includes a pair of TALENs), Ttago system and/or CRISPR/Cas nuclease systems are used to cleave DNA at a mitochondrial genome, typically a mutant mitochondrial genome such that heteroplasmy (as between the wild-type and mutant mitochondrial genomes) is shifted and the amount of mutant mtDNA reduced. The target (e.g., mutant mtDNA) may be inactivated following cleavage because double-repair pathways in mtDNA are inefficient and, accordingly, selective cleavage of mutant mtDNA (where wild-type mtDNA is not cleaved) leads to rapid degradation of the mutant mtDNA and a corresponding shift in the heteroplasmic ratio of wild-type to mutant mtDNA. The nucleases described herein can induce a double-stranded (DSB) or single-stranded break (nick) in the target DNA. In some embodiments, two nickases are used to create a DSB by introducing two nicks. In some cases, the nickase is a ZFN, while in others, the nickase is a TALEN or a CRISPR/Cas nickase. Any of the nucleases described herein (e.g., ZFNs, TALENs, CRISPR/Cas etc.) may specifically target mutant mtDNA, including for instance the target sequences shown Table 2, including for example a target site comprising 9 to 20 or more (9, 10, 11, 12, 13, 14, 15, 16, 17, 18, 19, 20 or more) contiguous or non-contiguous nucleotides of the wild-type or mutant sequences. Any mutant may be targeted by the DNA-binding domain, including but not limited to m.5024C>T. For example, a non-limiting set of human diseases associated with mtDNA mutations includes Kearns-Sayre syndrome (KSS; progressive myopathy, ophthalmoplegia, cardiomyopathy); CPEO: chronic progressive external ophthalmoplegia; and Pearson Syndrome (pancytopenia, lactic acidosis) where all three are associated with single large deletions (approximately 5 kb) in the mitochondrial genome. Other human diseases associated with mutant mitochondria are MELAS (myopathy, encephalopathy, lactic acidosis, and stroke-like episodes) tied to the 3243 A>G mutations (also referred to herein as A3243G or 3243G) and/or 3271 T>C mutations (also referred to herein as T3271C or 3271C) in the TRNL1 gene or sporadic mutations in ND1 and ND5; MERRF (myoclonic epilepsy with ragged red fibers, myopathy) associated with the 8344 A>G and/or 8356 T>C mutations (referred to herein as A8344G or 8344G/T8356C or 8356C) in the TRNK gene; NARP (neuropathy, ataxia, retinitis pigmentosa) associated with the 8993 T>G mutation (referred to herein as T8993G or 8993G) in the ATP6 gene; MILS (progressive brain-stem disorder, also known as Maternally Inherited Leigh Syndrome) also associated with the 8993 T>G/C mutation (referred to herein as T8993G, T8993C, 8993G, 8993C) and/or 9176 T>G/C mutation (referred to herein as T9176G, T9176C, 9176G, 9176C) in ATP6; MIDD (diabetes, deafness) associated with the 3243 A>G mutation (referred to herein as A3243G or 3243G) in the TRNL1 gene; LHON (optic neuropathy) associated with 3460 G>A mutation (referred to herein as G3460A or 3460A) in ND1, 11778 G>A mutation (referred to herein as G11778A or 11778A) in ND4, and/or a 14484 T>C mutation (referred to herein as T14484C or 14484C) in the ND6 gene; myopathy and diabetes associated with a 14709 T>C mutation (referred to herein as T14709C or 14709C) in the TRNE gene; sensorineural hearing loss and deafness associated with the 1555 A>G mutation (referred to herein as A1555G or 1555G) in the RNR1 gene and sporadic mutations in the TRNS1 gene; exercise intolerance tied to sporadic mutations in the CYB gene; and fatal, infantile encephalopathy Leigh/Leigh-like syndrome associated with 10158 T>C (referred to as T10158C or 10158C) and/or 10191 T>C (referred to as T10191C or 10191C) mutations and/or 10197 G>A mutation (referred to as G10197A or 10197A) in the ND3 gene. Other mutations in mtDNA include the 14709 T>C mutation (referred to as T14709C or 14709C) in the ND6 gene; 14459 G>A and/or 14487 T>C mutations (referred to herein as G14459A or 14459A and T14487C or 14487C) in the ND6 gene and/or 11777 C>A mutation (referred to as C11777A or 11777A) in the ND4 gene and/or 1624 C>T mutation (referred to as C1624T or 1624T) associated with Leigh Syndrome; 13513 G>A mutation (referred to as G13513A or 13513A) in the ND5 gene; 7445 A>G mutation (referred to as A7445G or 7445G) and/or insertion at 7472 associated with deafness and myopathy; 5545 C>T mutation (referred to as C5545T or 5545T) associated with multisystem disorder; and 4300 A>G mutation (referred to as A4300G or 4300G) associated with cardiomyopathy. See, e.g., Greaves and Taylor (2006) IUBMB Life 58(3): 143-151; Taylor and Turnbull (2005) Nat Rev Genet 6(5): 389-402; and Tuppen et al (2010) ibid). All mutations are numbered relative to the wild-type sequence.

In one aspect, described herein is a non-naturally occurring zinc-finger protein (ZFP) that binds to a target site in a mtDNA genome, wherein the ZFP comprises one or more engineered zinc-finger binding domains. In one embodiment, the ZFP is a zinc-finger nuclease (ZFN) that cleaves a target genomic region of interest, wherein the ZFN comprises one or more engineered zinc-finger binding domains and a nuclease cleavage domain or cleavage half-domain. Cleavage domains and cleavage half domains can be obtained, for example, from various restriction endonucleases and/or homing endonucleases and may be wild-type or engineered (mutant). In one embodiment, the cleavage half-domains are derived from a Type IIS restriction endonuclease (e.g., FokI). In certain embodiments, the zinc finger domain a zinc finger protein with the recognition helix domains ordered as shown in a single row of Table 1. Nucleases comprising these zinc finger proteins may include any linker sequence (e.g., linking it to the cleavage domain) and any cleavage domain (e.g., a dimerization mutant such as an ELD mutant; a FokI domain having mutation at one or more of 416, 422, 447, 448, and/or 525; and/or catalytic domain mutants that result in nickase functionality). See, e.g., U.S. Pat. Nos. 8,703,489; 9,200,266; 8,623,618; and 7,914,796; and U.S. Patent Publication No. 20180087072. In certain embodiments, the ZFP of the ZFN binds to a target site of 9 to 18 or more nucleotides within the sequence shown in Table 2. In certain embodiments, the ZFN selectively binds to a mutant mtDNA (as compared to wild-type mtDNA) such that the ZFN selectively cleaves mutant mtDNA (as compared to cleavage of wild-type mtDNA). In further embodiments, the ZFN selectively binds to a target site in mutant mtDNA comprising one or more of the following mutations: 1555G, 1624T, 3243G, 3460A, 3271C, 4300G, 5545T, 7445G, 7472 random insertions, 8344G, 8356C 8993G, 9176G/C, 10158C, 10191C, 10197A, 11777A, 11778A, 13513A, 14459A, 14484C, 14487C, or 14709C, numbered relative to the wild-type sequence, where the nucleotide following the position indicates the mutant sequence. Any of the ZFNs described herein may include a pair of ZFNs (e.g., left and right) in which one member of the pair binds to mutant mtDNA and one member of the pair binds to wild-type mtDNA. Alternatively, the ZFNs described herein may include a pair of ZFNs (left and right) in which both ZFNs bind to wild-type mtDNA or both ZFNs bind to mutant mtDNA.

In another aspect, described herein is a Transcription Activator Like Effector (TALE) protein that binds to target site (e.g., a target site comprising at least 9 or 12 (e.g., 9 to 20 or more) nucleotides of a target sequence as shown in Table 2 in a mtDNA, wherein the TALE comprises one or more engineered TALE binding domains. In one embodiment, the TALE is a nuclease (TALEN) that cleaves a target genomic region of interest, wherein the TALEN comprises one or more engineered TALE DNA binding domains and a nuclease cleavage domain or cleavage half-domain. Cleavage domains and cleavage half domains can be obtained, for example, from various restriction endonucleases and/or homing endonucleases (meganuclease). In one embodiment, the cleavage half-domains are derived from a Type IIS restriction endonuclease (e.g., FokI). In other embodiments, the cleavage domain is derived from a meganuclease, which meganuclease domain may also exhibit DNA-binding functionality. In certain embodiments, the TALEN selectively binds to a mutant mtDNA (as compared to wild-type mtDNA) such that the TALEN selectively cleaves mutant mtDNA (as compared to cleavage of wild-type mtDNA). In further embodiments, the TALEN selectively binds to target sites comprising the following mutations: 1555G, 1624T, 3243G, 3460A, 3271C, 4300G, 5545T, 7445G, 7472 random insertions, 8344G, 8356C 8993G, 9176G/C, 10158C, 10191C, 10197A, 11777A, 11778A, 13513A, 14459A, 14484C, 14487C, or 14709C, numbered relative to the wild-type sequence, where the nucleotide following the position indicates the mutant sequence. Any of the TALENs described herein may include a pair of TALENs (e.g., left and right) in which one member of the pair binds to mutant mtDNA and one member of the pair binds to wild-type mtDNA. Alternatively, the TALENs as described herein may include a pair of TALENs (left and right) in which both TALENs bind to wild-type mtDNA or both TALENs bind to mutant mtDNA.

In another aspect, described herein is a CRISPR/Cas system that binds to target site in mtDNA, wherein the CRISPR/Cas system comprises one or more engineered single guide RNA or a functional equivalent, as well as a Cas9 nuclease. In certain embodiments, the single guide RNA (sgRNA) binds to a sequence comprising 9, 12 or more contiguous nucleotides of a target site as shown in Table 2. In certain embodiments, the sgRNA selectively binds to a mutant mtDNA (as compared to wild-type mtDNA) such that the CRISPR/Cas nuclease selectively cleaves mutant mtDNA (as compared to cleavage of wild-type mtDNA). In further embodiments, the CRISPR/Cas system selectively binds to target sites comprising the following mutations: 1555G, 1624T, 3243G, 3460A, 3271C, 4300G, 5545T, 7445G, 7472 random insertions, 8344G, 8356C 8993G, 9176G/C, 10158C, 10191C, 10197A, 11777A, 11778A, 13513A, 14459A, 14484C, 14487C, or 14709C, numbered relative to the wild-type sequence, where the nucleotide following the position indicates the mutant sequence. Any of the sgRNAs described herein may bind to selectively to mutant, or alternatively, wild-type mtDNA. In cases in which a pair of sgRNAs are used, one or both members may bind to wild-type or mutant mtDNA.

The nucleases (e.g., ZFN, CRISPR/Cas system, Ttago and/or TALEN) as described herein may bind to and/or cleave the region of interest in a coding or non-coding region of mtDNA, such as, for example, a leader sequence, trailer sequence or intron, or within a non-transcribed region, either upstream or downstream of the coding region. The target site may be 9-18 or more nucleotides in length including a target site as shown Table 2 or a target site encompassing 1555G, 1624T, 3243G, 3460A, 3271C, 4300G, 5545T, 7445G, 7472 random insertions, 8344G, 8356C 8993G, 9176G/C, 10158C, 10191C, 10197A, 11777A, 11778A, 13513A, 14459A, 14484C, 14487C, or 14709C in mtDNA. In certain embodiments, the DNA-binding domain (ZFP, TALE, sgRNA, etc.) of the nuclease selectively binds to mutant mtDNA (as compared to cleavage of wild-type mtDNA). In some embodiments, the DNA binding domain of the nuclease(s) binds to a selected location in the TRNL1, ND1, ND5, TRNK, ATP6, ND4, ND6, TRNE, RNR1, TRNS, CYB, CYTb, 12SrRNA and/or ND3 mitochondrial genes.

In another aspect, described herein are one or more polynucleotides encoding one or more nucleases (e.g., ZFNs, CRISPR/Cas systems, Ttago and/or TALENs described herein). In certain embodiments, the same polynucleotide encodes one nuclease (e.g., both left and right monomers of a paired nuclease or all components of a CRISPR/Cas system) while in other embodiments, separate polynucleotides are used for the components of the nuclease (e.g., a first polynucleotide encoding one member (e.g., the left member/monomer) of a paired nuclease and a second polynucleotide encoding the other member (e.g., the right member/monomer) of a paired nuclease. The polynucleotide may be formulated in a viral or non-viral vector, including but not limited to AAV, Ad, retroviral vectors or the like as well as mRNA, plasmids, minicircle DNA and the like. In certain embodiments, the vector is targeted to a specific tissue or organ, for example an AAV vector targeted to the heart (cardiac tissue). In certain embodiments, the nuclease is a ZFN comprising left and right ZFNs, formulated separately as AAV vector compositions and administered concurrently (e.g., formulated as a single pharmaceutical composition comprising both AAV vectors).

In another aspect, described herein is a ZFN, CRISPR/Cas system, Ttago and/or TALEN expression vector comprising a polynucleotide, encoding one or more nucleases (e.g., ZFNs, CRISPR/Cas systems, Ttago and/or TALENs) as described herein, operably linked to a promoter. In one embodiment, the expression vector is a viral vector (e.g., an AAV vector). In one aspect, the viral vector exhibits tissue specific tropism.

In another aspect, described herein is a host cell comprising one or more nuclease (e.g., ZFN, CRISPR/Cas systems, Ttago and/or TALEN) expression vectors.

In another aspect, pharmaceutical compositions comprising an expression vector (e.g., comprising one or more components of one or more nucleases) as described herein are provided. In some embodiments, the pharmaceutical composition may comprise more than one expression vector. In some embodiments, the pharmaceutical composition comprises a first expression vector comprising a first polynucleotide, and a second expression vector comprising a second polynucleotide. In some embodiments, the first polynucleotide and the second polynucleotide are different. In some embodiments, the first polynucleotide and the second polynucleotide are substantially the same. In certain embodiments, the pharmaceutical composition comprises a first AAV vector encoding a left monomer of a ZFN pair and/or a second AAV vector encoding a right monomer of the ZFN pair. In certain embodiments, the concentration of the pharmaceutical compositions (e.g., a pharmaceutical comprising a polynucleotide such as an AAV vector including one or both monomers) is between 1×10¹⁰ to 1×10¹⁴ (or any value therebetween) vector genomes (vg) per cell or subject. In some embodiments, the concentration of the pharmaceutical composition is 1×10¹², 5×10¹² or 1×10¹³ vg per cell or subject (e.g., by tail-vein injection). The pharmaceutical compositions are suitable for delivery to a subject, including but not limited to systemic, intraperitoneal, intravenous, intramuscular, mucosal or topical delivery methods of combinations thereof. The pharmaceutical composition may further comprise a donor sequence (e.g., a transgene encoding a protein lacking or deficient in a disease or disorder such as mitochondrial disorder). In some embodiments, the donor sequence is associated with an expression vector.

In some embodiments, a fusion protein comprising a DNA-binding domain (e.g., zinc finger protein or TALE or sgRNA or meganuclease) and a wild-type or engineered cleavage domain or cleavage half-domain are provided.

In another aspect, described herein are compositions comprising one or more of the nucleases (e.g., ZFNs, TALENs, TtAgo and/or CRISPR/Cas systems) described herein, including a nuclease comprising a DNA-binding molecule (e.g., ZFP, TALE, sgRNA, etc.) and a nuclease (cleavage) domain. In certain embodiments, the composition comprises one or more nucleases in combination with a pharmaceutically acceptable excipient. In some embodiments, the composition comprises two or more sets (pairs) of nucleases, each set with different specificities. In other aspects, the composition comprises different types of nucleases. In some embodiments, the composition comprises polynucleotides encoding mtDNA-specific nucleases, while in other embodiments, the composition comprises mtDNA-specific nuclease proteins. In certain embodiments, the compositions are suitable for delivery to a subject, including via systemic delivery.

In another aspect, described herein is a polynucleotide encoding one or more nucleases or nuclease components (e.g., ZFNs, TALENs, TtAgo or nuclease domains of the CRISPR/Cas system) described herein. The polynucleotide may be, for example, mRNA or DNA. In some aspects, the mRNA may be chemically modified (See e.g. Kormann et al., (2011) Nature Biotechnology 29(2):154-157). In other aspects, the mRNA may comprise an ARCA cap (see U.S. Pat. Nos. 7,074,596; and 8,153,773). In further embodiments, the mRNA may comprise a mixture of unmodified and modified nucleotides (see U.S. Patent Publication No. 2012/0195936). In another aspect, described herein is a nuclease expression vector comprising a polynucleotide, encoding one or more ZFNs, TALENs, TtAgo or CRISPR/Cas systems described herein, operably linked to a promoter. In one embodiment, the expression vector is a viral vector, for example an AAV vector.

In another aspect, described herein is a host cell comprising one or more nucleases, one or more nuclease expression vectors as described herein. In certain embodiments, the host cell comprises in which the amount of mutant mtDNA is reduced or eliminated, thereby shifting the heteroplasmic ratio of mtDNA in the cell (as compared to a wild-type cell). In certain embodiments, the heteroplasmic ratio is shifted at least 5% or more, preferably at least 10% or more, and even more preferably at least 20% or more in favor of wild-type (non-mutant mtDNA). The host cell may be stably transformed or transiently transfected or any combination thereof with one or more nuclease expression vectors. In other embodiments, the one or more nuclease expression vectors express one or more nucleases in the host cell. In another embodiment, the host cell may further comprise an exogenous polynucleotide donor sequence. In any of the embodiments, described herein, the host cell can comprise an embryo cell, for example a one or more mouse, rat, rabbit or other mammalian cell embryo (e.g., a non-human primate). In some embodiments, the host cell comprises a tissue. Also described are cells or cell lines produced or descended from the cells described herein, including pluripotent, totipotent, multipotent or differentiated cells comprising a modification in mtDNA (e.g., heteroplasmic ratio of mtDNA). In certain embodiments, described herein are differentiated cells as described herein comprising a modification as described herein, which differentiated cells are descended from a stem cell as described herein. In certain embodiments, the host cell is a cardiac cell or a stem cell, for example a hematopoietic stem cell or an induced pluripotent stem cell.

In another aspect, described herein is a method for cleaving mtDNA gene in a cell, the method comprising: (a) introducing, into the cell, one or more polynucleotides encoding one or more nucleases that target mtDNA under conditions such that the nuclease(s) is(are) expressed and the mtDNA is cleaved. In certain embodiments, mutant mtDNA is selectively cleaved as compared to wild-type mtDNA. This results in a shift in the heteroplasmic ratio of mutant mtDNA:wild-type mtDNA. Optionally, the methods further comprise administering a donor (e.g., therapeutic protein) to the cell, which may be integrated into the cell's genome or into mtDNA. Integration of one or more donor molecule(s) occurs via homology-directed repair (HDR) or by non-homologous end joining (NHEJ) associated repair. Furthermore, the nuclease-encoding polynucleotide(s) and/or donors may be introduced into the cell using any one or combinations of delivery systems (e.g., non-viral vector, LNP or viral vector). In certain embodiments a vector that is specific for a certain cell, tissue and/or organ type is used, for example an AAV vector that is specific for cardiac tissue, brain tissue, lung tissue, muscle tissue or the like. In certain embodiments, cleavage of the mutant mtDNA shifts heteroplasmy toward the wild-type (e.g., including partial or complete restoration to wild-type sequences) sequence, thereby treating and/or preventing mitochondrial disease in a subject in need thereof. In certain embodiments the mutant mtDNA cleaved and restored to wild-type comprises a point mutation (e.g., 5024C>T).

In any of the compositions or methods described herein, the one or more polynucleotides can be provided and/or delivered at any concentration (dose) that provides the desired effect. In preferred embodiments, the one or more polynucleotides are delivered using an adeno-associated virus (AAV) vector at 10,000 1×10¹⁴ or more vector genome per cell or subject (or any value therebetween). In certain embodiments, the one or more polynucleotides are delivered using a lentiviral vector at MOI between 250 and 1,000 (or any value therebetween). In other embodiments, the one or more polynucleotides are delivered using a plasmid vector at 150-1,500 ng/100,000 cells (or any value therebetween). In other embodiments, the one or more polynucleotides are delivered as mRNA at 150-1,500 ng/100,000 cells (or any value therebetween). When two or more polynucleotides are delivered, the vectors may be the same or different vectors and the same vectors may be delivered in any ratio, including but not limited to a 1:1 ratio. In certain embodiments, two AAV vectors are used to deliver the components of a paired nuclease (e.g., ZFN comprising MTM25 monomer and WTM1 monomer) at any concentration per monomer, including but not limited to 1×10¹⁰ to 1×10¹⁴ (or any value therebetween), optionally at 5×10¹² vg/monomer. In certain embodiments, the dose of individual monomers, or alternatively, the total dose (both monomers) is 1×10¹², 5×10¹² or 1×10¹³ vg per cell or subject (e.g., by tail-vein injection). In some embodiments, the ZFN are given a total AAV dose of 5e12 vg/kg (for example 2.5 e12 vg/kg of each AAV-ZFN monomer); a total AAV dose of 1e13 vg/kg (for example 0.5e13 vg/kg of each AAV-ZFN monomer); a total AAV dose of 5e13 vg/kg (for example 2.5e13 vg/kg of each AAV-ZFN monomer); a total AAV dose of 1e14 vg/kg (for example 0.5e14 vg/kg of each AAV-ZFN monomer); a total AAV dose of 5e14 vg/kg (for example 2.5e14 vg/kg of each AAV-ZFN monomer); or a total AAV dose of 1e15 vg/kg (for example 0.5e15 vg/kg of each AAV-ZFN monomer). In certain embodiments, the AAV is administered by intravenous injection.

In yet another aspect, provided herein is a cell comprising genetically modified mtDNA, for example a cell in which the heteroplasmic ratio of wild-type to mutant mtDNA is altered by reducing and/or eliminating mutant mtDNA in the cell. In certain embodiments, the cell heteroplasmic ratio is reduced as compared to a cell from a subject with a mitochondrial disorder. The mutant mtDNA is reduced and/or eliminated from the cell by degradation following cleavage of mutant mtDNA by a nuclease specific for the mutant form of mtDNA (e.g., a nuclease targeted to a sequence of 9-20 or more base pairs as shown in Table 2 or encompassing one or more of the following mutations: 1555G, 1624T, 3243G, 3460A, 3271C, 4300G, 5545T, 7445G, 7472 random insertions, 8344G, 8356C 8993G, 9176G/C, 10158C, 10191C, 10197A, 11777A, 11778A, 13513A, 14459A, 14484C, 14487C, or 14709C. Cleavage that precipitates degradation may be within the target site(s) and/or cleavage site(s) and/or within 1-50 base pairs of edge of a target site of 9-18 or more base pairs of the target sequences. The modified cells as described herein may be isolated or may be within a subject, for example a subject with a mitochondrial disorder.

In any of the methods and compositions described herein, the cells may be any eukaryotic cell. In certain embodiments, the cells are differentiated cells, for example, cardiac cells, brain cells, liver cells, kidney cells, muscle cells, nerve cells, cells of the gut, cells of the eye and/or cells of the ear etc. In other embodiments, the cells are stem cells. In other embodiments, the cells are patient-derived, for example autologous CD34+ (hematopoietic) stem cells (e.g., mobilized in patients from the bone marrow into the peripheral blood via granulocyte colony-stimulating factor (GCSF) administration). The CD34+ cells can be harvested, purified, cultured, and the nucleases introduced into the cell by any suitable method.

In another aspect, the methods and compositions of the invention provide for the use of compositions (nucleases, pharmaceutical compositions, polynucleotides, expression vectors, cells, cell lines and/or animals such as transgenic animals) as described herein, for example for use in treatment and/or prevention of a mitochondrial disease. In certain embodiments, these compositions are used in the screening of drug libraries and/or other therapeutic compositions (i.e., antibodies, structural RNAs, etc.) for use in treatment of mitochondrial disorders. Such screens can begin at the cellular level with manipulated cell lines or primary cells, and can progress up to the level of treatment of a whole animal (e.g., veterinary or human therapy). Thus, in certain aspects, described herein is a method of treating and/or preventing mitochondrial disease in a subject in need thereof, the method comprising administering one or more nucleases, polynucleotides and/or cells as described herein to the subject. The methods may be ex vivo or in vivo. In certain embodiments, a cell as described herein is administered to the subject. In any of the methods described herein, the cell may be a stem cell derived from the subject (patient-derived stem cell).

In any of the compositions and methods described herein, the nucleases are introduced in mRNA form and/or using one or more non-viral, LNP or viral vector(s). In certain embodiments, the nuclease(s) are introduced in mRNA form. In other embodiments, the nuclease(s) is(are) introduced using a viral vector, for instance an adeno-associated vector (AAV) including AAV1, AAV3, AAV4, AAV5, AAV6, AAV8, AAV 8.2, AAV9, AAV rh10, AAV2/8, AAV2/5 and AAV2/6, or via a lentiviral or integration-defective lentiviral vector.

Once delivered to the cell, the nuclease(s) is transcribed and/or translated, and the nuclease proteins are taken up by the mitochondria. Thus, in some embodiments, the nuclease(s) comprise a mitochondrial targeting peptide (see e.g. U.S. Pat. No. 9,139,628; Omuta (1998) J. Biochem 123(6): 1010-6). In certain embodiments, a tissue- or cell-specific vector is used, for example a vector that is specific for the heart (cardiac tissue).

Any cell can be modified using the compositions and methods of the invention, including but not limited to prokaryotic or eukaryotic cells such as bacterial, insect, yeast, fish, mammalian (including non-human mammals), and plant cells. In certain embodiments, the cell is a cardiac cell, a brain cell, a liver cell, a spleen cell, an intestinal cell, or an immune cell, for example a T-cell (e.g., CD4+, CD3+, CD8+, etc.), a dendritic cell, a B cell or the like. In other embodiments, the cell is a pluripotent, totipotent or multipotent stem cell, for example an induced pluripotent stem cell (iPSC), hematopoietic stem cells (e.g., CD34+), an embryonic stem cell or the like. Specific stem cell types that may be used with the methods and compositions of the invention include embryonic stem cells (ESC), induced pluripotent stem cells (iPSC) and hematopoietic stem cells (e.g., CD34+ cells). The iPSCs can be derived from patient samples and/or from normal controls wherein the patient derived iPSC can be mutated to the normal or wild type gene sequence at the gene of interest, or normal cells can be altered to the known disease allele at the gene of interest. Similarly, the hematopoietic stem cells can be isolated from a patient or from a donor.

Thus, described herein are methods and compositions for altering mtDNA genomes, including but not limited to, selective cleavage of mutant mtDNA to alter the heteroplasmic ratio of mutant and wild-type mtDNA in cell, organ and/or tissue (e.g., of a subject in need thereof), thereby treating and/or preventing mitochondrial disease. The compositions and methods can be for use in vitro, in vivo or ex vivo, and comprise administering an artificial transcription factor or nuclease that includes a DNA-binding domain targeted to mtDNA.

A kit, comprising the nucleic acids, nucleases and/or cells of the invention, is also provided. The kit may comprise nucleic acids encoding the nucleases, (e.g. RNA molecules or ZFN, TALEN, TtAgo or CRISPR/Cas system encoding genes contained in a suitable expression vector), or aliquots of the nuclease proteins, donor molecules, suitable stemness modifiers, cells, instructions for performing the methods of the invention, and the like.

These and other aspects will be readily apparent to the skilled artisan in light of disclosure as a whole.

### BRIEF DESCRIPTION OF THE DRAWINGS

FIGS. 1A through 1G depict design of nucleases targeted to mitochondrial DNA and in vivo mtDNA heteroplasmy modification. FIG. 1A are schematics showing the monomer “WTM1” that bound to sequences (SEQ ID NO:1) upstream of m.5024 in wild-type and mutant genomes and the mutant specific monomer “MTM25” that bound preferentially to the mutated site (SEQ ID NO:2) due to the C>T mutation in the target site (indicated by the *). Dimerization of the obligatory heterodimeric FokI domains produced DNA double-stand breaks resulting in specific depletion of mutant mtDNA. FIG. 1B depicts schematics of the libraries used for screening nucleases targeted to mouse mtDNA (left panel) and of the screening assay (right panel). For screening, the MTM(n) mtZFN library (labeled “MTM(n)”) was cloned into a backbone containing ribosome stuttering T2A site (“2A”), the WTM1 mtZFN (“WTM1”) and hammerhead ribozyme (“HHR”) such that the backbone also co-expressed mCherry from a separate promoter (SV40). These constructs were transfected into mouse embryonic fibroblasts (MEFs) bearing m.5024C>T, transfectants were sorted by fluorescence activated cell storing (FACS) at 24 hours; DNA extracted and heteroplasmy shifting in the transfected fibroblasts determined by pyrosequencing. FIG. 1C shows results of pyrosequencing analysis of m.5024C>T heteroplasmy from MEFs transfected with controls or MTM25/WTM1 at differing concentrations facilitated by tetracycline-sensitive HHR 7. Change (“A m.5024C>T (%)”) in m.5024C>T heteroplasmy was plotted according to the different conditions tested. “utZFN” are mtZFNs that do not have a target site in mouse mtDNA 7. n=4-8. Error bars indicate SD. Statistical analysis performed: two-tailed Student's t-test ***p<0.01. FIG. 1D is a schematic depicting in vivo experiments. MTM25 and WTM1 were encoded in separate AAV genomes that are encapsidated in AAV9.45 then simultaneously systemically (tail vein) administered. Animals were sacrificed at 65 days post-injection. FIG. 1E shows Western blot analysis of total heart protein from animals injected with MTM25 and/or WTM1. Both proteins include the HA tag and are differentiated by molecular weight. FIG. 1E shows pyrosequencing analysis of m.5024C>T heteroplasmy from ear and heart total DNA. Change (Δ) in m.5024C>T between these is plotted. n=4-20 (Table S1). Error bars indicate SEM. Statistical analysis performed: two-tailed Student's t-test. ***p<0.001. FIG. 1F shows analysis of mtDNA copy number by qPCR. Each square indicates one animal. n=4-8 (Table S1). Error bars indicate SEM. Statistical analysis performed: two-tailed Student's t-test **p<0.01.

FIGS. 2A through 2E depict reduction of m.5024C>T mtDNA heteroplasmy results in phenotype rescue in live subjects. FIG. 2A is an illustration of mt-tRNA:ALA that is encoded by the m.5024C>T mutation. The location of the mutant ‘A’ inserted due to the 5024 C>T mutation is indicated by the circle. Given the nature and position of this mutation, transcribed tRNA molecules containing the mutation mispairing are unlikely to fold correctly or be aminoacylated, resulting in reduced steady-state levels of mt-tRNA:ALA at high levels of m.5024C>T heteroplasmy. FIG. 2B shows quantification of northern blot analysis of total heart RNA extracts. mt-tRNA abundance is normalized to 5S rRNA. n=4-6. Error bars indicate SEM. Statistical analysis performed: two-tailed Student's t-test. ***p<0.001. The data indicated an increase in the presence of the mt tRNA:ALA as normalized to mt tRNA:CYS in cells treated with the WTM1/MTM25 ZFN pair as compared to the untreated cells. FIG. 2C depicts principal component analysis (PCA) plot of metabolomic data for intermediate dose (5e12 vg/animal) AAV treated mice and age/initial heteroplasmy-matched (vehicle treated) controls used to assess the physiological effects of the mt-tRNA^{ALA} molecular phenotype rescue. Each square indicates one animal (see Example 2). FIG. 2D shows measurements of total metabolite abundance (phosphoenol pyruvate in left graph; pyruvate in middle graph; lactate in right graph) from mouse heart tissue of intermediate dose AAV treated mice (right bars “+AAV”) and age/initial heteroplasmy-matched controls (left bars “VEH”) by LC/MS. Chemical structures of terminal glycolytic metabolites, and reactions linking these, are depicted in the top panel. Error bars indicate SEM. Statistical analysis performed: one-tailed Student's t-test. *p<0.05. FIG. 2E shows chemical structures (top panel) and in vivo abundance of the initial reactant and products of the glycolytic pathway from mouse cardiac tissue. Elevated glucose levels (left graph), coupled with diminished downstream metabolite abundance (glucose-6-phosphate shown in middle graph and frustose-6-phosphase shown in right graph) in treated animal hearts contributes to the profile of mitochondrial metabolic recovery and enhancement of aerobic glycolysis observed in treated animals (right bars “+AAV”) when compared with controls (left bars “VEH”).

### DETAILED DESCRIPTION

Disclosed herein are compositions and methods for targeted modification of mtDNA, including selective cleavage of mutant mtDNA such that heteroplasmy in mtDNA is shifted and a reversion of molecular and biochemical phenotypes to wild-type is achieved.

The invention contemplates genetic modification to mtDNA, including but not limited to selective cleavage of mutant mtDNA for the treatment and/or prevention of mitochondrial diseases of any genetic origin in a subject in need thereof. Any mutant mtDNA may be targeted by the DNA-binding domain, including but not limited to m.5024C>T, 1555G, 1624T, 3243G, 3460A, 3271C, 4300G, 5545T, 7445G, 7472 random insertions, 8344G, 8356C 8993G, 9176G/C, 10158C, 10191C, 10197A, 11777A, 11778A, 13513A, 14459A, 14484C, 14487C and/or 14709C.

### General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al., MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolfe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

### Definitions

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acids.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (K_{d}) of 10⁻⁶M⁻¹ or lower. “Affinity” refers to the strength of binding: increased binding affinity being correlated with a lower K_{d}.

A “binding domain” is a molecule that is able to bind non-covalently to another molecule. A binding molecule can bind to, for example, a DNA molecule (a DNA-binding protein such as a zinc finger protein or TAL-effector domain protein or a single guide RNA), an RNA molecule (an RNA-binding protein) and/or a protein molecule (a protein-binding protein). In the case of a protein-binding molecule, it can bind to itself (to form homodimers, homotrimers, etc.) and/or it can bind to one or more molecules of a different protein or proteins. A binding molecule can have more than one type of binding activity. For example, zinc finger proteins have DNA-binding, RNA-binding and protein-binding activity. Thus, DNA-binding molecules, including DNA-binding components of artificial nucleases and transcription factors include but are not limited to, ZFPs, TALEs and sgRNAs.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP. Artificial nucleases and transcription factors can include a ZFP DNA-binding domain and a functional domain (nuclease domain for a ZFN or transcriptional regulatory domain for ZFP-TF). The term “zinc finger nuclease” includes one ZFN as well as a pair of ZFNs (including first and second ZFNs also known as left and right ZFNs) that dimerize to cleave the target gene.

A “TALE DNA binding domain” or “TALE” is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. See, e.g., U.S. Pat. No. 8,586,526. Artificial nucleases and transcription factors can include a TALE DNA-binding domain and a functional domain (nuclease domain for a TALEN or transcriptional regulatory domain for TALEN-TF). The term “TALEN” includes one TALEN as well as a pair of TALENs (including first and second TALENs also known as left and right TALENs) that dimerize to cleave the target gene.

Zinc finger and TALE binding domains can be “engineered” to bind to a predetermined nucleotide sequence, for example via engineering (altering one or more amino acids) of the recognition helix region of a naturally occurring zinc finger or TALE protein. Therefore, engineered DNA binding proteins (zinc fingers or TALEs) are proteins that are non-naturally occurring. Non-limiting examples of methods for engineering DNA-binding proteins are design and selection. A designed DNA binding protein is a protein not occurring in nature whose design/composition results principally from rational criteria. Rational criteria for design include application of substitution rules and computerized algorithms for processing information in a database storing information of existing ZFP and/or TALE designs and binding data. See, for example, U.S. Pat. Nos. 6,140,081; 6,453,242; 6,534,261; and 8,585,526; see also International Patent Publication Nos. WO 98/53058; WO 98/53059; WO 98/53060; WO 02/016536; and WO 03/016496.

A “selected” zinc finger protein or TALE is a protein not found in nature whose production results primarily from an empirical process such as phage display, interaction trap or hybrid selection. See e.g., U.S. Pat. Nos. 5,789,538; 5,925,523; 6,007,988; 6,013,453; 6,200,759; 8,586,526; and International Patent Publication Nos. WO 95/19431; WO 96/06166; WO 98/53057; WO 98/54311; WO 00/27878; WO 01/60970; WO 01/88197; and WO 02/099084.

“TtAgo” is a prokaryotic Argonaute protein thought to be involved in gene silencing. TtAgo is derived from the bacteria Thermus thermophilus. See, e.g., Swarts et al., ibid, G. Sheng et al., (2013) Proc. Natl. Acad. Sci. U.S.A. 111, 652). A “TtAgo system” is all the components required including, for example, guide DNAs for cleavage by a TtAgo enzyme.

“Recombination” refers to a process of exchange of genetic information between two polynucleotides, including but not limited to, donor capture by non-homologous end joining (NHEJ) and homologous recombination. For the purposes of this disclosure, “homologous recombination (HR)” refers to the specialized form of such exchange that takes place, for example, during repair of double-strand breaks in cells via homology-directed repair mechanisms. This process requires nucleotide sequence homology, uses a “donor” molecule to template repair of a “target” molecule (i.e., the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized HR often results in an alteration of the sequence of the target molecule such that part or all of the sequence of the donor polynucleotide is incorporated into the target polynucleotide.

In the methods of the disclosure, one or more targeted nucleases as described herein create a double-stranded break (DSB) in the target sequence (e.g., cellular chromatin) at a predetermined site. The DSB may result in deletions and/or insertions by homology-directed repair or by non-homology-directed repair mechanisms. Deletions may include any number of base pairs. Similarly, insertions may include any number of base pairs including, for example, integration of a “donor” polynucleotide, optionally having homology to the nucleotide sequence in the region of the break. The donor sequence may be physically integrated or, alternatively, the donor polynucleotide is used as a template for repair of the break via homologous recombination, resulting in the introduction of all or part of the nucleotide sequence as in the donor into the cellular chromatin. Thus, a first sequence in cellular chromatin can be altered and, in certain embodiments, can be converted into a sequence present in a donor polynucleotide. Thus, the use of the terms “replace” or “replacement” can be understood to represent replacement of one nucleotide sequence by another, (i.e., replacement of a sequence in the informational sense), and does not necessarily require physical or chemical replacement of one polynucleotide by another.

In any of the methods described herein, additional pairs of zinc-finger proteins, TALENs, TtAgo or CRISPR/Cas systems can be used for additional double-stranded cleavage of additional target sites within the cell.

Any of the methods described herein can be used for insertion of a donor of any size and/or partial or complete inactivation of one or more target sequences in a cell by targeted integration of donor sequence that disrupts expression of the gene(s) of interest. Cell lines with partially or completely inactivated genes are also provided.

In any of the methods described herein, the exogenous nucleotide sequence (the “donor sequence” or “transgene”) can contain sequences that are homologous, but not identical, to genomic sequences in the region of interest, thereby stimulating homologous recombination to insert a non-identical sequence in the region of interest. Thus, in certain embodiments, portions of the donor sequence that are homologous to sequences in the region of interest exhibit between about 80 to 99% (or any integer therebetween) sequence identity to the genomic sequence that is replaced. In other embodiments, the homology between the donor and genomic sequence is higher than 99%, for example if only 1 nucleotide differs as between donor and genomic sequences of over 100 contiguous base pairs. In certain cases, a non-homologous portion of the donor sequence can contain sequences not present in the region of interest, such that new sequences are introduced into the region of interest. In these instances, the non-homologous sequence is generally flanked by sequences of 50-1,000 base pairs (or any integral value therebetween) or any number of base pairs greater than 1,000, that are homologous or identical to sequences in the region of interest. In other embodiments, the donor sequence is non-homologous to the first sequence, and is inserted into the genome by non-homologous recombination mechanisms.

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

A “cleavage half-domain” is a polypeptide sequence which, in conjunction with a second polypeptide (either identical or different) forms a complex having cleavage activity (preferably double-strand cleavage activity). The terms “first and second cleavage half-domains;” “+ and − cleavage half-domains” and “right and left cleavage half-domains” are used interchangeably to refer to pairs of cleavage half-domains that dimerize.

An “engineered cleavage half-domain” is a cleavage half-domain that has been modified so as to form obligate heterodimers with another cleavage half-domain (e.g., another engineered cleavage half-domain). See, also, U.S. Pat. Nos. 8,623,618; 7,888,121; 7,914,796; and 8,034,598, incorporated herein by reference in their entireties.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 100,000,000 nucleotides in length (or any integer value therebetween or thereabove), preferably between about 100 and 100,000 nucleotides in length (or any integer therebetween), more preferably between about 2000 and 20,000 nucleotides in length (or any value therebetween) and even more preferable, between about 5 and 15 kb (or any value therebetween).

“Chromatin” is the nucleoprotein structure comprising the cellular genome. Cellular chromatin comprises nucleic acid, primarily DNA, and protein, including histones and non-histone chromosomal proteins. The majority of eukaryotic cellular chromatin exists in the form of nucleosomes, wherein a nucleosome core comprises approximately 150 base pairs of DNA associated with an octamer comprising two each of histones H2A, H2B, H3 and H4; and linker DNA (of variable length depending on the organism) extends between nucleosome cores. A molecule of histone H1 is generally associated with the linker DNA. For the purposes of the present disclosure, the term “chromatin” is meant to encompass all types of cellular nucleoprotein, both prokaryotic and eukaryotic. Cellular chromatin includes both chromosomal and episomal chromatin.

A “chromosome,” is a chromatin complex comprising all or a portion of the genome of a cell. The genome of a cell is often characterized by its karyotype, which is the collection of all the chromosomes that comprise the genome of the cell. The genome of a cell can comprise one or more chromosomes.

An “episome” is a replicating nucleic acid, nucleoprotein complex or other structure comprising a nucleic acid that is not part of the chromosomal karyotype of a cell. Examples of episomes include plasmids and certain viral genomes.

An “accessible region” is a site in cellular chromatin in which a target site present in the nucleic acid can be bound by an exogenous molecule which recognizes the target site. Without wishing to be bound by any particular theory, it is believed that an accessible region is one that is not packaged into a nucleosomal structure. The distinct structure of an accessible region can often be detected by its sensitivity to chemical and enzymatic probes, for example, nucleases.

A “target site” or “target sequence” is a nucleic acid sequence that defines a portion of a nucleic acid to which a binding molecule will bind, provided sufficient conditions for binding exist. Target sites may be any length, for example, 9 to 20 or more nucleotides and length and the bound nucleotides may be contiguous or non-contiguous.

An “exogenous” molecule is a molecule that is not normally present in a cell but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer. An exogenous molecule can also be the same type of molecule as an endogenous molecule but derived from a different species than the cell is derived from. For example, a human nucleic acid sequence may be introduced into a cell line originally derived from a mouse or hamster.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

As used herein, the term “product of an exogenous nucleic acid” includes both polynucleotide and polypeptide products, for example, transcription products (polynucleotides such as RNA) and translation products (polypeptides).

A “fusion” molecule is a molecule in which two or more subunit molecules are linked, preferably covalently. The subunit molecules can be the same chemical type of molecule, or can be different chemical types of molecules. Examples of the first type of fusion molecule include, but are not limited to, fusion proteins (for example, a fusion between a ZFP or TALE DNA-binding domain and one or more activation domains) and fusion nucleic acids (for example, a nucleic acid encoding the fusion protein described supra). Examples of the second type of fusion molecule include, but are not limited to, a fusion between a triplex-forming nucleic acid and a polypeptide, and a fusion between a minor groove binder and a nucleic acid.

Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are presented elsewhere in this disclosure.

A “gene,” for the purposes of the present disclosure, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” of gene expression refers to a change in the activity of a gene. Modulation of expression can include, but is not limited to, gene activation and gene repression. Genome editing (e.g., cleavage, alteration, inactivation, random mutation) can be used to modulate expression. Gene inactivation refers to any reduction in gene expression as compared to a cell that does not include a ZFP, TALE, TtAgo or CRISPR/Cas system as described herein. Thus, gene inactivation may be partial or complete.

A “region of interest” is any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

“Eukaryotic” cells include, but are not limited to, fungal cells (such as yeast), plant cells, animal cells, mammalian cells and human cells (e.g., T-cells), including stem cells (pluripotent and multipotent).

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

With respect to fusion polypeptides, the term “operatively linked” can refer to the fact that each of the components performs the same function in linkage to the other component as it would if it were not so linked. For example, with respect to a fusion polypeptide in which a ZFP, TALE, TtAgo or Cas DNA-binding domain is fused to an activation domain, the ZFP, TALE, TtAgo or Cas DNA-binding domain and the activation domain are in operative linkage if, in the fusion polypeptide, the ZFP, TALE, TtAgo or Cas DNA-binding domain portion is able to bind its target site and/or its binding site, while the activation domain is able to upregulate gene expression. When a fusion polypeptide in which a ZFP, TALE, TtAgo or Cas DNA-binding domain is fused to a cleavage domain, the ZFP, TALE, TtAgo or Cas DNA-binding domain and the cleavage domain are in operative linkage if, in the fusion polypeptide, the ZFP, TALE, TtAgo or Cas DNA-binding domain portion is able to bind its target site and/or its binding site, while the cleavage domain is able to cleave DNA in the vicinity of the target site.

A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA-binding function of a polypeptide can be determined, for example, by filter-binding, electrophoretic mobility-shift, or immunoprecipitation assays. DNA cleavage can be assayed by gel electrophoresis. See Ausubel et al., supra. The ability of a protein to interact with another protein can be determined, for example, by co-immunoprecipitation, two-hybrid assays or complementation, both genetic and biochemical. See, for example, Fields et al., (1989) Nature 340:245-246; U.S. Pat. No. 5,585,245 and International Patent Publication No. WO 98/44350.

A “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors.

The terms “subject” and “patient” are used interchangeably and refer to mammals such as human patients and non-human primates, as well as experimental animals such as rabbits, dogs, cats, rats, mice, and other animals. Accordingly, the term “subject” or “patient” as used herein means any mammalian patient or subject to which the nucleases, donors and/or genetically modified cells of the invention can be administered. Subjects of the present invention include those with a disorder.

“Sternness” refers to the relative ability of any cell to act in a stem cell-like manner, i.e., the degree of toti-, pluri-, or oligopotentcy and expanded or indefinite self-renewal that any particular stem cell may have.

An “ACTR” is an Antibody-coupled T-cell Receptors that is an engineered T cell component capable of binding to an exogenously supplied antibody. The binding of the antibody to the ACTR component arms the T cell to interact with the antigen recognized by the antibody, and when that antigen is encountered, the ACTR comprising T cell is triggered to interact with antigen (see U.S. Patent Publication No. 2015/0139943).

### Fusion Molecules

Described herein are compositions, for example nucleases, that are useful for cleavage of a selected target gene in mtDNA in a cell.

Recombinant transcription factors comprising the DNA binding domains from zinc finger proteins (“ZFPs”) or TAL-effector domains (“TALEs”) and engineered nucleases including zinc finger nucleases (“ZFNs”), TALENs, CRISPR/Cas nuclease systems, and homing endonucleases that are all designed to specifically bind to target DNA sites have the ability to regulate gene expression of endogenous genes and are useful in genome engineering, gene therapy and treatment of mitochondrial disorders. See, e.g., U.S. Pat. Nos. 9,394,545; 9,150,847; 9,206,404; 9,045,763; 9,005,973; 8,956,828; 8,936,936; 8,945,868; 8,871,905; 8,586,526; 8,563,314; 8,329,986; 8,399,218; 6,534,261; 6,599,692; 6,503,717; 6,689,558; 7,067,317; 7,262,054; 7,888,121; 7,972,854; 7,914,796; 7,951,925; 8,110,379; 8,409,861; U.S. Patent Publication Nos. 2003/0232410; 2005/0208489; 2005/0026157; 2005/0064474; 2006/0063231; 2008/0159996; 2010/0218264; 2012/0017290; 2011/0265198; 2013/0137104; 2013/0122591; 2013/0177983; 2013/0177960; and 2015/0056705, the disclosures of which are incorporated by reference in their entireties for all purposes. Further, targeted nucleases are being developed based on the Argonaute system (e.g., from T. thermophilus, known as TtAgo′, see Swarts et al., (2014) Nature 507(7491): 258-261), which also may have the potential for uses in genome editing and gene therapy.

Nuclease-mediated gene therapy can be used to genetically engineer a cell to have one or more inactivated genes and/or to cause that cell to express a product not previously being produced in that cell (e.g., via transgene insertion and/or via correction of an endogenous sequence). Examples of uses of transgene insertion include the insertion of one or more genes encoding one or more novel therapeutic proteins, insertion of a coding sequence encoding a protein that is lacking in the cell or in the individual, insertion of a wild-type gene in a cell containing a mutated gene sequence, and/or insertion of a sequence that encodes a structural nucleic acid such as shRNA or siRNA. Examples of useful applications of ‘correction’ of an endogenous gene sequence include alterations of disease-associated gene mutations, shifts in heteroplasmy, alterations in sequences encoding splice sites, alterations in regulatory sequences and targeted alterations of sequences encoding structural characteristics of a protein. Transgene constructs can be inserted by either homology directed repair (HDR) or by end capture during non-homologous end joining (NHEJ) driven processes. See, e.g., U.S. Pat. Nos. 9,045,763; 9,005,973; 7,888,121; and 8,703,489.

Clinical trials using these engineered transcription factors and nucleases have shown that these molecules are capable of treating various conditions, including cancers, HIV and/or blood disorders (such as hemoglobinopathies and/or hemophilias). See, e.g., Yu et al., (2006) FASEB J. 20:479-481; Tebas et al., (2014) New Eng J Med 370(10):901. Thus, these approaches can be used for the treatment of diseases.

In certain embodiments, one or more components of the fusion molecules (e.g., nucleases) are naturally occurring. In other embodiments, one or more of the components of the fusion molecules (e.g., nucleases) are non-naturally occurring, i.e., engineered in the DNA-binding molecules and/or cleavage domain(s). For example, the DNA-binding portion of a naturally-occurring nuclease may be altered to bind to a selected target site (e.g., a single guide RNA of a CRISPR/Cas system or a meganuclease that has been engineered to bind to site different than the cognate binding site). In other embodiments, the nuclease comprises heterologous DNA-binding and cleavage domains (e.g., zinc finger nucleases; TAL-effector domain DNA binding proteins; meganuclease DNA-binding domains with heterologous cleavage domains). Thus, any nuclease may be used in the practice of the present invention including but not limited to, at least one ZFN, TALEN, meganuclease, CRISPR/Cas nuclease or the like, which nucleases that cleave a target gene, which cleavage results in genomic modification of the target gene (e.g., insertions and/or deletions into the cleaved gene).

Also described herein are methods to increase specificity of cleavage activity through independent titration of the engineered cleavage half-domain partners of a nuclease complex. In some embodiments, the ratio of the two partners (half cleavage domains) is given at a 1:2, 1:3, 1:4, 1:5, 1:6, 1:8, 1:9, 1:10 or 1:20 ratio, or any value therebetween. In other embodiments, the ratio of the two partners is greater than 1:30. In other embodiments, the two partners are deployed at a ratio that is chosen to be different from 1:1. When used individually or in combination, the methods and compositions of the invention provide surprising and unexpected increases in targeting specificity via reductions in off-target cleavage activity. The nucleases used in these embodiments may comprise ZFNs, TALENs, CRISPR/Cas, CRISPR/dCas and TtAgo, or any combination thereof.

### A. DNA-Binding Molecules

The fusion molecules described herein can include any DNA-binding molecule (also referred to as DNA-binding domain), including protein domains and/or polynucleotide DNA-binding domains. In certain embodiments, the DNA-binding domain binds to a target site of 9-18 or more nucleotides, in which the target site comprises one or more mutant mtDNA sequences. The mutation may be a point mutation, for example a target site that includes the m.5024C>T mutation.

In certain embodiments, the composition and methods described herein employ a meganuclease (homing endonuclease) DNA-binding domain for binding to the donor molecule and/or binding to the region of interest in the genome of the cell. Naturally-occurring meganucleases recognize 15-40 base-pair cleavage sites and are commonly grouped into four families: the LAGLIDADG family, the GIY-YIG family, the His-Cyst box family and the HNH family. Exemplary homing endonucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII. Their recognition sequences are known. See also U.S. Pat. Nos. 5,420,032 and 6,833,252; Belfort et al., (1997) Nucleic Acids Res. 25:3379-3388; Dujon et al., (1989) Gene 82:115-118; Perler et al., (1994) Nucleic Acids Res. 22, 1125-1127; Jasin (1996) Trends Genet. 12:224-228; Gimble et al., (1996)J Mol. Biol. 263:163-180; Argast et al., (1998) J. Mol. Biol. 280:345-353 and the New England Biolabs catalogue. In addition, the DNA-binding specificity of homing endonucleases and meganucleases can be engineered to bind non-natural target sites. See, for example, Chevalier et al., (2002) Molec. Cell 10:895-905; Epinat et al. (2003) Nucleic Acids Res. 31:2952-2962; Ashworth et al., (2006) Nature 441:656-659; Paques et al., (2007) Current Gene Therapy 7:49-66; and U.S. Patent Publication No. 2007/0117128. The DNA-binding domains of the homing endonucleases and meganucleases may be altered in the context of the nuclease as a whole (i.e., such that the nuclease includes the cognate cleavage domain) or may be fused to a heterologous cleavage domain.

In other embodiments, the DNA-binding domain of one or more of the nucleases used in the methods and compositions described herein comprises a naturally occurring or engineered (non-naturally occurring) TAL effector DNA binding domain. See, e.g., U.S. Pat. No. 8,586,526, incorporated by reference in its entirety herein. The plant pathogenic bacteria of the genus Xanthomonas are known to cause many diseases in important crop plants. Pathogenicity of Xanthomonas depends on a conserved type III secretion (T3S) system which injects more than 25 different effector proteins into the plant cell. Among these injected proteins are transcription activator-like (TAL) effectors which mimic plant transcriptional activators and manipulate the plant transcriptome (see Kay et al., (2007) Science 318:648-651). These proteins contain a DNA binding domain and a transcriptional activation domain. One of the most well characterized TAL-effectors is AvrBs3 from Xanthomonas campestgris pv. Vesicatoria (see Bonas et al., (1989) Mol Gen Genet 218: 127-136 and International Patent Publication No. WO 2010/079430). TAL-effectors contain a centralized domain of tandem repeats, each repeat containing approximately 34 amino acids, which are key to the DNA binding specificity of these proteins. In addition, they contain a nuclear localization sequence and an acidic transcriptional activation domain (for a review see Schornack et al. (2006) J Plant Physiol 163(3): 256-272). In addition, in the phytopathogenic bacteria Ralstonia solanacearum two genes, designated brg11 and hpx17 have been found that are homologous to the AvrBs3 family of Xanthomonas in the R. solanacearum biovar 1 strain GMI1000 and in the biovar 4 strain RS1000 (See Heuer et al. (2007) Appl and Envir Micro 73(13): 4379-4384). These genes are 98.9% identical in nucleotide sequence to each other but differ by a deletion of 1,575 bp in the repeat domain of hpx17. However, both gene products have less than 40% sequence identity with AvrBs3 family proteins of Xanthomonas. See, e.g., U.S. Pat. No. 8,586,526, incorporated by reference in its entirety herein.

Specificity of these TAL effectors depends on the sequences found in the tandem repeats. The repeated sequence comprises approximately 102 bp and the repeats are typically 91-100% homologous with each other (Bonas et al., ibid). Polymorphism of the repeats is usually located at positions 12 and 13 and there appears to be a one-to-one correspondence between the identity of the hypervariable diresidues (RVD) at positions 12 and 13 with the identity of the contiguous nucleotides in the TAL-effector's target sequence (see Moscou and Bogdanove, (2009) Science 326:1501 and Boch et al., (2009) Science 326:1509-1512). Experimentally, the natural code for DNA recognition of these TAL-effectors has been determined such that an HD sequence at positions 12 and 13 leads to a binding to cytosine (C), NG binds to T, NI to A, C, G or T, NN binds to A or G, and ING binds to T. These DNA binding repeats have been assembled into proteins with new combinations and numbers of repeats, to make artificial transcription factors that are able to interact with new sequences and activate the expression of a non-endogenous reporter gene in plant cells (Boch et al., ibid). Engineered TAL proteins have been linked to a FokI cleavage half domain to yield a TAL effector domain nuclease fusion (TALEN). See, e.g., U.S. Pat. No. 8,586,526; Christian et al. (2010) Genetics epub 10.1534/genetics.110.120717). In certain embodiments, TALE domain comprises an N-cap and/or C-cap as described in U.S. Pat. No. 8,586,526.

In certain embodiments, the DNA binding domain of one or more of the nucleases used for in vivo cleavage and/or targeted cleavage of the genome of a cell comprises a zinc finger protein. Preferably, the zinc finger protein is non-naturally occurring in that it is engineered to bind to a target site of choice. See, for example, See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al., (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

An engineered zinc finger binding domain can have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, co-owned U.S. Pat. Nos. 6,453,242 and 6,534,261, incorporated by reference herein in their entireties.

Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as International Patent Publication Nos. WO 98/37186; WO 98/53057; WO 00/27878; WO 01/88197; and GB Patent No. 2,338,237. In addition, enhancement of binding specificity for zinc finger binding domains has been described, for example, in co-owned International Patent Publication No. WO 02/077227.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein.

A ZFP can be operably associated (linked) to one or more nuclease (cleavage) domains to form a ZFN. The term “a ZFN” includes a pair of ZFNs that dimerize to cleave the target gene. Methods and compositions can also be used to increase the specificity of a ZFN, including a nuclease pair, for its intended target relative to other unintended cleavage sites, known as off-target sites (see U.S. Patent Publication No. 20180087072). Thus, nucleases described herein can comprise mutations in one or more of their DNA binding domain backbone regions and/or one or more mutations in their nuclease cleavage domains. These nucleases can include mutations to amino acid within the ZFP DNA binding domain (‘ZFP backbone’) that can interact non-specifically with phosphates on the DNA backbone, but they do not comprise changes in the DNA recognition helices. Thus, the invention includes mutations of cationic amino acid residues in the ZFP backbone that are not required for nucleotide target specificity. In some embodiments, these mutations in the ZFP backbone comprise mutating a cationic amino acid residue to a neutral or anionic amino acid residue. In some embodiments, these mutations in the ZFP backbone comprise mutating a polar amino acid residue to a neutral or non-polar amino acid residue. In preferred embodiments, mutations at made at position (−5), (−9) and/or position (−14) relative to the DNA binding helix. In some embodiments, a zinc finger may comprise one or more mutations at (−5), (−9) and/or (−14). In further embodiments, one or more zinc finger in a multi-finger zinc finger protein may comprise mutations in (−5), (−9) and/or (−14). In some embodiments, the amino acids at (−5), (−9) and/or (−14) (e.g. an arginine (R) or lysine (K)) are mutated to an alanine (A), leucine (L), Ser (S), Asp (N), Glu (E), Tyr (Y) and/or glutamine (Q).

In some aspects, the DNA-binding domain (e.g., ZFP, TALE, sgRNA, etc.) targets mutant mtDNA preferentially as compared to wild-type. In paired nuclease, one DNA-binding domain may target a wild-type sequence and the other DNA-binding domain may target a mutant sequence. Alternatively, both DNA-binding domains may target wild-type or mutant sequences. In certain embodiments, the DNA-binding domain targets sites (9 to 18 or more nucleotides) in mutant mtDNA (e.g., m.5024C>T) as shown in Table 2. In other embodiments, the DNA-binding domain targets sequences in mutant mtDNA comprising one or more of the following mutations: 1555G, 1624T, 3243G, 3460A, 3271C, 4300G, 5545T, 7445G, 7472 random insertions, 8344G, 8356C 8993G, 9176G/C, 10158C, 10191C, 10197A, 11777A, 11778A, 13513A, 14459A, 14484C, 14487C, or 14709C.

Selection of target sites; ZFPs and methods for design and construction of fusion proteins (and polynucleotides encoding same) are known to those of skill in the art and described in detail in U.S. Pat. Nos. 6,140,081; 5,789,538; 6,453,242; 6,534,261; 5,925,523; 6,007,988; 6,013,453; 6,200,759; and International Patent Publication Nos. WO 95/19431; WO 96/06166; WO 98/53057; WO 98/54311; WO 00/27878; WO 01/60970; WO 01/88197; WO 02/099084; WO 98/53058; WO 98/53059; WO 98/53060; WO 02/016536; and WO 03/016496.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein.

In certain embodiments, the DNA-binding molecule is part of a CRISPR/Cas nuclease system. See, e.g., U.S. Pat. No. 8,697,359 and U.S. Patent Publication No. 2015/0056705. The CRISPR (clustered regularly interspaced short palindromic repeats) locus, which encodes RNA components of the system, and the cas (CRISPR-associated) locus, which encodes proteins (Jansen et al. (2002) Mol. Microbiol. 43:1565-1575; Makarova et al. (2002) Nucleic Acids Res. 30:482-496; Makarova et al. (2006) Biol. Direct 1:7; Haft et al. (2005) PLoS Comput. Biol. 1:e60) make up the gene sequences of the CRISPR/Cas nuclease system. CRISPR loci in microbial hosts contain a combination of CRISPR-associated (Cas) genes as well as non-coding RNA elements capable of programming the specificity of the CRISPR-mediated nucleic acid cleavage.

The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called ‘adaptation’, (ii) expression of the relevant proteins, as well as expression and processing of the array, followed by (iii) RNA-mediated interference with the alien nucleic acid. Thus, in the bacterial cell, several of the so-called ‘Cas’ proteins are involved with the natural function of the CRISPR/Cas system and serve roles in functions such as insertion of the alien DNA etc.

In certain embodiments, Cas protein may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some case, the cell does not naturally produce Cas protein and is genetically engineered to produce a Cas protein. In some embodiments, the Cas protein is a small Cas9 ortholog for delivery via an AAV vector (Ran et al., (2015) Nature 510, p. 186).

In some embodiments, the DNA binding molecule is part of a TtAgo system (see Swarts et al., ibid; Sheng et al., ibid). In eukaryotes, gene silencing is mediated by the Argonaute (Ago) family of proteins. In this paradigm, Ago is bound to small (19-31 nt) RNAs. This protein-RNA silencing complex recognizes target RNAs via Watson-Crick base pairing between the small RNA and the target and endonucleolytically cleaves the target RNA (Vogel (2014) Science 344:972-973). In contrast, prokaryotic Ago proteins bind to small single-stranded DNA fragments and likely function to detect and remove foreign (often viral) DNA (Yuan et al. (2005) Mol. Cell 19, 405; Olovnikov et al. (2013) Mol. Cell 51, 594; Swarts et al., ibid). Exemplary prokaryotic Ago proteins include those from Aquifex aeolicus, Rhodobacter sphaeroides, and Thermus thermophilus.

One of the most well-characterized prokaryotic Ago protein is the one from T. thermophilus (TtAgo; Swarts et al., ibid). TtAgo associates with either 15 nt or 13-25 nt single-stranded DNA fragments with 5′ phosphate groups. This “guide DNA” bound by TtAgo serves to direct the protein-DNA complex to bind a Watson-Crick complementary DNA sequence in a third-party molecule of DNA. Once the sequence information in these guide DNAs has allowed identification of the target DNA, the TtAgo-guide DNA complex cleaves the target DNA. Such a mechanism is also supported by the structure of the TtAgo-guide DNA complex while bound to its target DNA (G. Sheng et al., ibid). Ago from Rhodobacter sphaeroides (RsAgo) has similar properties (Olovnikov et al., ibid).

Exogenous guide DNAs of arbitrary DNA sequence can be loaded onto the TtAgo protein (Swarts et al., ibid.). Since the specificity of TtAgo cleavage is directed by the guide DNA, a TtAgo-DNA complex formed with an exogenous, investigator-specified guide DNA will therefore direct TtAgo target DNA cleavage to a complementary investigator-specified target DNA. In this way, one may create a targeted double-strand break in DNA. Use of the TtAgo-guide DNA system (or orthologous Ago-guide DNA systems from other organisms) allows for targeted cleavage of genomic DNA within cells. Such cleavage can be either single- or double-stranded. For cleavage of mammalian genomic DNA, it would be preferable to use of a version of TtAgo codon optimized for expression in mammalian cells. Further, it might be preferable to treat cells with a TtAgo-DNA complex formed in vitro where the TtAgo protein is fused to a cell-penetrating peptide. Further, it might be preferable to use a version of the TtAgo protein that has been altered via mutagenesis to have improved activity at 37 degrees Celsius. Ago-RNA-mediated DNA cleavage could be used to affect a panoply of outcomes including gene knock-out, targeted gene addition, gene correction, targeted gene deletion using techniques standard in the art for exploitation of DNA breaks.

Thus, the nuclease comprises a DNA-binding molecule in that specifically binds to a target site in any gene into which it is desired to insert a donor (transgene).

### B. Cleavage Domains

Any suitable cleavage domain can be operatively linked to a DNA-binding domain to form a nuclease. For example, ZFP DNA-binding domains have been fused to nuclease domains to create ZFNs—a functional entity that is able to recognize its intended nucleic acid target through its engineered (ZFP) DNA binding domain and cause the DNA to be cut near the ZFP binding site via the nuclease activity, including for use in genome modification in a variety of organisms. See, for example, U.S. Pat. Nos. 7,888,121; 8,623,618; 7,888,121; 7,914,796; and 8,034,598; and U.S. Patent Publication No. 2011/0201055. Likewise, TALE DNA-binding domains have been fused to nuclease domains to create TALENs. See, e.g., U.S. Pat. No. 8,586,526.

As noted above, the cleavage domain may be heterologous to the DNA-binding domain, for example a zinc finger DNA-binding domain and a cleavage domain from a nuclease or a TALEN DNA-binding domain and a cleavage domain, or meganuclease DNA-binding domain and cleavage domain from a different nuclease. Heterologous cleavage domains can be obtained from any endonuclease or exonuclease. Exemplary endonucleases from which a cleavage domain can be derived include, but are not limited to, restriction endonucleases and homing endonucleases. Additional enzymes which cleave DNA are known (e.g., S1 Nuclease; mung bean nuclease; pancreatic DNase I; micrococcal nuclease; yeast HO endonuclease. One or more of these enzymes (or functional fragments thereof) can be used as a source of cleavage domains and cleavage half-domains.

Similarly, a cleavage half-domain can be derived from any nuclease or portion thereof, as set forth above, that requires dimerization for cleavage activity. In general, two fusion proteins are required for cleavage if the fusion proteins comprise cleavage half-domains. Alternatively, a single protein comprising two cleavage half-domains can be used. The two cleavage half-domains can be derived from the same endonuclease (or functional fragments thereof), or each cleavage half-domain can be derived from a different endonuclease (or functional fragments thereof). In addition, the target sites for the two fusion proteins are preferably disposed, with respect to each other, such that binding of the two fusion proteins to their respective target sites places the cleavage half-domains in a spatial orientation to each other that allows the cleavage half-domains to form a functional cleavage domain, e.g., by dimerizing. Thus, in certain embodiments, the near edges of the target sites are separated by 5-8 nucleotides or by 15-18 nucleotides. However, any integral number of nucleotides or nucleotide pairs can intervene between two target sites (e.g., from 2 to 50 nucleotide pairs or more). In general, the site of cleavage lies between the target sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme FokI catalyzes double-stranded cleavage of DNA, at 9 nucleotides from its recognition site on one strand and 13 nucleotides from its recognition site on the other. See, for example, U.S. Pat. Nos. 5,356,802; 5,436,150; and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982. Thus, in one embodiment, fusion proteins comprise the cleavage domain (or cleavage half-domain) from at least one Type IIS restriction enzyme and one or more zinc finger binding domains, which may or may not be engineered.

An exemplary Type IIS restriction enzyme, whose cleavage domain is separable from the binding domain, is FokI. This particular enzyme is active as a dimer. Bitinaite et al., (1998) Proc. Natl. Acad. Sci. USA 95:10,570-10,575. Accordingly, for the purposes of the present disclosure, the portion of the FokI enzyme used in the disclosed fusion proteins is considered a cleavage half-domain. Thus, for targeted double-stranded cleavage and/or targeted replacement of cellular sequences using zinc finger-FokI fusions, two fusion proteins, each comprising a FokI cleavage half-domain, can be used to reconstitute a catalytically active cleavage domain. Alternatively, a single polypeptide molecule containing a zinc finger binding domain and two FokI cleavage half-domains can also be used. Parameters for targeted cleavage and targeted sequence alteration using zinc finger—FokI fusions are provided elsewhere in this disclosure.

A cleavage domain or cleavage half-domain can be any portion of a protein that retains cleavage activity, or that retains the ability to multimerize (e.g., dimerize) to form a functional cleavage domain.

Exemplary Type IIS restriction enzymes are described in International Publication WO 07/014275, incorporated herein in its entirety. Additional restriction enzymes also contain separable binding and cleavage domains, and these are contemplated by the present disclosure. See, for example, Roberts et al., (2003) Nucleic Acids Res. 31:418-420.

In certain embodiments, the cleavage domain comprises one or more engineered cleavage half-domain (also referred to as dimerization domain mutants) that minimize or prevent homodimerization, as described, for example, in U.S. Pat. Nos. 8,623,618; 7,888,121; 7,914,796; and 8,034,598; and U.S. Publication No. 2011/0201055, the disclosures of all of which are incorporated by reference in their entireties herein. Amino acid residues at positions 446, 447, 479, 483, 484, 486, 487, 490, 491, 496, 498, 499, 500, 531, 534, 537, and 538 of FokI are all targets for influencing dimerization of the FokI cleavage half-domains.

In certain embodiments, the engineered cleavage half domains are derived from FokI and comprise one or more mutations in one or more of amino acid residues 416, 422, 447, 448, and/or 525 (see, e.g., U.S. Patent Publication No. 20180087072) numbered relative to the wild-type FokI cleavage half-domain (residues 394 to 579 of full length FokI) as shown below:

Wild type FokI cleavage half domain
(SEQ ID NO: 1)
QLVKSELEEKKSELRHKLKYVPHEYIELIEIARNSTQDRILEMKVMEFFM
KVYGYRGKHLGGSRKPDGAIYTVGSPIDYGVIVDTKAYSGGYNLPIGQAD
EMQRYVEENQTRNKHINPNEWWKVYPSSVTEFKFLFVSGHFKGNYKAQLT
RLNHITNCNGAVLSVEELLIGGEMIKAGTLTLEEVRRKFNNGEINF

These mutations decrease the non-specific interaction between the FokI domain and a DNA molecule. In other embodiments the cleavage half domains derived from FokI comprises a mutation in one or more of amino acid residues 414-426, 443-450, 467-488, 501-502, and/or 521-531. The mutations may include mutations to residues found in natural restriction enzymes homologous to FokI. In certain embodiments, the mutations are substitutions, for example substitution of the wild-type residue with a different amino acid, for example serine (S), e.g. R416S or K525S. In a preferred embodiment, the mutation at positions 416, 422, 447, 448 and/or 525 comprise replacement of a positively charged amino acid with an uncharged or a negatively charged amino acid. In another embodiment, the engineered cleavage half domain comprises mutations in amino acid residues 499, 496 and 486 in addition to the mutations in one or more amino acid residues 416, 422, 447, 448, or 525. In a preferred embodiment, the invention provides fusion proteins wherein the engineered cleavage half-domain comprises a polypeptide in which the wild-type Gln (Q) residue at position 486 is replaced with a Glu (E) residue, the wild-type Ile (I) residue at position 499 is replaced with a Leu (L) residue and the wild-type Asn (N) residue at position 496 is replaced with an Asp (D) or a Glu (E) residue (“ELD” or “ELE”) in addition to one or more mutations at positions 416, 422, 447, 448, or 525.

Cleavage domains with more than one mutation may be used, for example mutations at positions 490 (E→K) and 538 (I→K) in one cleavage half-domain to produce an engineered cleavage half-domain designated “E490K:I538K” and by mutating positions 486 (Q→E) and 499 (I→L) in another cleavage half-domain to produce an engineered cleavage half-domain designated “Q486E:I499L;” mutations that replace the wild type Gln (Q) residue at position 486 with a Glu (E) residue, the wild type Iso (I) residue at position 499 with a Leu (L) residue and the wild-type Asn (N) residue at position 496 with an Asp (D) or Glu (E) residue (also referred to as a “ELD” and “ELE” domains, respectively); engineered cleavage half-domain comprising mutations at positions 490, 538 and 537 (numbered relative to wild-type FokI), for instance mutations that replace the wild type Glu (E) residue at position 490 with a Lys (K) residue, the wild type Iso (I) residue at position 538 with a Lys (K) residue, and the wild-type His (H) residue at position 537 with a Lys (K) residue or a Arg (R) residue (also referred to as “KKK” and “KKR” domains, respectively); and/or engineered cleavage half-domain comprises mutations at positions 490 and 537 (numbered relative to wild-type FokI), for instance mutations that replace the wild type Glu (E) residue at position 490 with a Lys (K) residue and the wild-type His (H) residue at position 537 with a Lys (K) residue or a Arg (R) residue (also referred to as “KIK” and “KIR” domains, respectively). See, e.g., U.S. Pat. Nos. 7,914,796; 8,034,598; and 8,623,618, the disclosures of which are incorporated by reference in its entirety for all purposes. In other embodiments, the engineered cleavage half domain comprises the “Sharkey” and/or “Sharkey” mutations (see Guo et al., (2010) J Mol. Biol. 400(1):96-107).

Alternatively, nucleases may be assembled in vivo at the nucleic acid target site using so-called “split-enzyme” technology (see, e.g. U.S. Patent Publication No. 2009/0068164). Components of such split enzymes may be expressed either on separate expression constructs, or can be linked in one open reading frame where the individual components are separated, for example, by a self-cleaving 2A peptide or IRES sequence. Components may be individual zinc finger binding domains or domains of a meganuclease nucleic acid binding domain.

Nucleases can be screened for activity prior to use, for example in a yeast-based chromosomal system as described in U.S. Pat. No. 8,563,314.

The Cas9 related CRISPR/Cas system comprises two RNA non-coding components: tracrRNA and a pre-crRNA array containing nuclease guide sequences (spacers) interspaced by identical direct repeats (DRs). To use a CRISPR/Cas system to accomplish genome engineering, both functions of these RNAs must be present (see Cong et al. (2013) Sciencexpress 1/10.1126/science 1231143). In some embodiments, the tracrRNA and pre-crRNAs are supplied via separate expression constructs or as separate RNAs. In other embodiments, a chimeric RNA is constructed where an engineered mature crRNA (conferring target specificity) is fused to a tracrRNA (supplying interaction with the Cas9) to create a chimeric cr-RNA-tracrRNA hybrid (also termed a single guide RNA). (see Jinek et al. (2012) Science 337:816-821; Jinek et al. (2013) eLife 2:e00471. DOI: 10.7554/eLife.00471 and Cong, ibid).

In some embodiments, the CRISPR-Cpf1 system is used. The CRISPR-Cpf1 system, identified in Francisella spp, is a class 2 CRISPR-Cas system that mediates robust DNA interference in human cells. Although functionally conserved, Cpf1 and Cas9 differ in many aspects including in their guide RNAs and substrate specificity (see Fagerlund et al. (2015) Genom Bio 16:251). A major difference between Cas9 and Cpf1 proteins is that Cpf1 does not utilize tracrRNA, and thus requires only a crRNA. The FnCpf1 crRNAs are 42-44 nucleotides long (19-nucleotide repeat and 23-25-nucleotide spacer) and contain a single stem-loop, which tolerates sequence changes that retain secondary structure. In addition, the Cpf1 crRNAs are significantly shorter than the ˜100-nucleotide engineered sgRNAs required by Cas9, and the PAM requirements for FnCpf1 are 5′-TTN-3 and 5′-CTA-3′ on the displaced strand. Although both Cas9 and Cpf1 make double strand breaks in the target DNA, Cas9 uses its RuvC- and HNH-like domains to make blunt-ended cuts within the seed sequence of the guide RNA, whereas Cpf1 uses a RuvC-like domain to produce staggered cuts outside of the seed. Because Cpf1 makes staggered cuts away from the critical seed region, NHEJ will not disrupt the target site, therefore ensuring that Cpf1 can continue to cut the same site until the desired HDR recombination event has taken place. Thus, in the methods and compositions described herein, it is understood that the term ‘“Cas” includes both Cas9 and Cpf1 proteins. Thus, as used herein, a “CRISPR/Cas system” refers both CRISPR/Cas and/or CRISPR/Cpf1 systems, including both nuclease and/or transcription factor systems.

### Target Sites

As described in detail above, DNA-binding domains can be engineered to bind to any sequence of choice. An engineered DNA-binding domain can have a novel binding specificity, compared to a naturally-occurring DNA-binding domain.

The nuclease(s) can target any wild-type or mutant mtDNA sequence in certain embodiments, the nuclease selectively target(s) mutant mtDNA, for example a target site of 9-25 or more nucleotides (contiguous or non-contiguous) encompassing a mutant mtDNA sequence such as 1555G, 1624T, 3243G, 3460A, 3271C, 4300G, 5545T, 7445G, 7472 random insertions, 8344G, 8356C 8993G, 9176G/C, 10158C, 10191C, 10197A, 11777A, 11778A, 13513A, 14459A, 14484C, 14487C, or 14709C or a target site as shown in Table 2.

Construction of such expression cassettes, following the teachings of the present specification, utilizes methodologies well known in the art of molecular biology (see, for example, Ausubel or Maniatis). Before use of the expression cassette to generate a transgenic animal, the responsiveness of the expression cassette to the stress-inducer associated with selected control elements can be tested by introducing the expression cassette into a suitable cell line (e.g., primary cells, transformed cells, or immortalized cell lines).

Furthermore, although not required for expression, exogenous sequences may also transcriptional or translational regulatory sequences, for example, promoters, enhancers, insulators, internal ribosome entry sites, sequences encoding 2A peptides, hammerhead ribozymes, targeting peptides and/or polyadenylation signals. Further, the control elements of the genes of interest can be operably linked to reporter genes to create chimeric genes (e.g., reporter expression cassettes).

Targeted insertion of non-coding nucleic acid sequence may also be achieved. Sequences encoding antisense RNAs, RNAi, shRNAs and micro RNAs (miRNAs) may also be used for targeted insertions.

In additional embodiments, the donor nucleic acid may comprise non-coding sequences that are specific target sites for additional nuclease designs. Subsequently, additional nucleases may be expressed in cells such that the original donor molecule is cleaved and modified by insertion of another donor molecule of interest. In this way, reiterative integrations of donor molecules may be generated allowing for trait stacking at a particular locus of interest or at a safe harbor locus.

### Cells

Thus, provided herein are genetically modified cells comprising a genetically modified mtDNA gene. In certain embodiments the modification comprises cleavage of a mutant mtDNA such that the heteroplasmic ratio mtDNA is altered. In certain embodiments, the mutant mtDNA is cleaved in a patient with one or more mitochondrial disorders such that the disorder or symptoms associated therewith is treated and/or prevented. The nuclease may differentially bind and cleave any mutant mtDNA, including but not limited to binding at point mutations such as m.5024C>T.

Unlike random cleavage, targeted cleavage ensures that the mutant form of mtDNA is cleaved preferentially as compared to wild-type, for example when the nuclease is designed such that the DNA-binding domain binds to the mutated sequence and exhibits specificity for the mutant form.

Any cell type can be genetically modified as described herein, including but not limited to cells and cell lines. Other non-limiting examples of cells containing modified mtDNA include heart cells, brain cells, lung cells, liver cells, T-cells (e.g., CD4+, CD3+, CD8+, etc.); dendritic cells; B-cells; autologous (e.g., patient-derived) or heterologous pluripotent, totipotent or multipotent stem cells (e.g., CD34+ cells, induced pluripotent stem cells (iPSCs), embryonic stem cells or the like). In certain embodiments, the cells as described herein are CD34+ cells derived from a patient.

The cells as described herein are useful in treating and/or preventing mitochondrial disease in a subject with the disorder, for example, by in vivo or ex vivo therapies. For ex vivo therapies, nuclease-modified cells can be expanded and then reintroduced into the patient using standard techniques. See, e.g., Tebas et al., (2014) New Eng J Med 370(10):901. In the case of stem cells, after infusion into the subject, in vivo differentiation of these precursors into cells expressing mtDNA with altered heteroplasmic ratios as compared to wild-type (diseased) cells are produced. Pharmaceutical compositions comprising the cells as described herein are also provided. In addition, the cells may be cryopreserved prior to administration to a patient.

The cells and ex vivo methods as described herein provide treatment and/or prevention of a disorder (e.g., mitochondrial disorder) in a subject (e.g., a mammalian subject) and eliminate the need for continuous prophylactic pharmaceutical administration or risky procedures such as allogeneic bone marrow transplants or gamma retroviral delivery. As such, the invention described herein provides a safer, cost-effective and time efficient way of treating and/or preventing mitochondrial disorders.

### Delivery

The nucleases, polynucleotides encoding these nucleases, donor polynucleotides and compositions comprising the proteins and/or polynucleotides described herein may be delivered by any suitable means. In certain embodiments, the nucleases and/or donors are delivered in vivo. In other embodiments, the nucleases and/or donors are delivered to isolated cells (e.g., autologous or heterologous stem cells) for the provision of modified cells useful in ex vivo delivery to patients.

Methods of delivering nucleases as described herein are described, for example, in U.S. Pat. Nos. 6,453,242; 6,503,717; 6,534,261; 6,599,692; 6,607,882; 6,689,558; 6,824,978; 6,933,113; 6,979,539; 7,013,219; and 7,163,824, the disclosures of all of which are incorporated by reference herein in their entireties.

Nucleases and/or donor constructs as described herein may also be delivered using any nucleic acid delivery mechanism, including naked DNA and/or RNA (e.g., mRNA) and vectors containing sequences encoding one or more of the components. Any vector systems may be used including, but not limited to, plasmid vectors, DNA minicircles, retroviral vectors, lentiviral vectors, adenovirus vectors, poxvirus vectors; herpesvirus vectors and adeno-associated virus vectors, etc., and combinations thereof. See, also, U.S. Pat. Nos. 6,534,261; 6,607,882; 6,824,978; 6,933,113; 6,979,539; 7,013,219; and 7,163,824; and U.S. Patent Publication No. 2014/0335063, incorporated by reference herein in their entireties. Furthermore, it will be apparent that any of these systems may comprise one or more of the sequences needed for treatment. Thus, when one or more nucleases and a donor construct are introduced into the cell, the nucleases and/or donor polynucleotide may be carried on the same delivery system or on different delivery mechanisms. When multiple systems are used, each delivery mechanism may comprise a sequence encoding one or multiple nucleases and/or donor constructs (e.g., mRNA encoding one or more nucleases and/or mRNA or AAV carrying one or more donor constructs).

Conventional viral and non-viral based gene transfer methods can be used to introduce nucleic acids encoding nucleases and donor constructs in cells (e.g., mammalian cells) and target tissues. Non-viral vector delivery systems include DNA plasmids, DNA minicircles, naked nucleic acid, and nucleic acid complexed with a delivery vehicle such as a liposome or poloxamer. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bohm (eds.) (1995); and Yu et al., Gene Therapy 1:13-26 (1994).

Methods of non-viral delivery of nucleic acids include electroporation, lipofection, microinjection, biolistics, virosomes, liposomes, immunoliposomes, polycation or lipid:nucleic acid conjugates, lipid nanoparticles (LNP), naked DNA, naked RNA, capped RNA, artificial virions, and agent-enhanced uptake of DNA. Sonoporation using, e.g., the Sonitron 2000 system (Rich-Mar) can also be used for delivery of nucleic acids.

Additional exemplary nucleic acid delivery systems include those provided by Amaxa Biosystems (Cologne, Germany), Maxcyte, Inc. (Rockville, Md.), BTX Molecular Delivery Systems (Holliston, Mass.) and Copernicus Therapeutics Inc. (see for example U.S. Pat. No. 6,008,336). Lipofection is described in e.g., U.S. Pat. Nos. 5,049,386; 4,946,787; and 4,897,355) and lipofection reagents are sold commercially (e.g., Transfectam™ and Lipofectin™) Cationic and neutral lipids that are suitable for efficient receptor-recognition lipofection of polynucleotides include those of Felgner, International Patent Publication Nos. WO 91/17424, WO 91/16024. In some aspects, the nucleases are delivered as mRNAs and the transgene is delivered via other modalities such as viral vectors, minicircle DNA, plasmid DNA, single-stranded DNA, linear DNA, liposomes, nanoparticles and the like.

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183; 4,217,344; 4,235,871; 4,261,975; 4,485,054; 4,501,728; 4,774,085; 4,837,028; and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (see MacDiarmid et al., (2009) Nature Biotechnology 27(7):643).

The use of RNA or DNA viral based systems for the delivery of nucleic acids encoding engineered CRISPR/Cas systems take advantage of highly evolved processes for targeting a virus to specific cells in the body and trafficking the viral payload to the nucleus. Viral vectors can be administered directly to subjects (in vivo) or they can be used to treat cells in vitro and the modified cells are administered to subjects (ex vivo). Conventional viral based systems for the delivery of CRISPR/Cas systems include, but are not limited to, retroviral, lentivirus, adenoviral, adeno-associated, vaccinia and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (see, e.g., Buchscher et al. (1992) J. Virol. 66:2731-2739; Johann et al. (1992) J. Virol. 66:1635-1640; Sommerfelt et al. (1990) Virol. 176:58-59; Wilson et al. (1989) J. Virol. 63:2374-2378; Miller et al. (1991) J. Virol. 65:2220-2224; International Patent Publication No. WO 1994/026877).

In applications in which transient expression is preferred, adenoviral based systems can be used. Adenoviral based vectors are capable of very high transduction efficiency in many cell types and do not require cell division. With such vectors, high titer and high levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; International Patent Publication No. WO 93/24641; Kotin (1994) Human Gene Therapy 5:793-801; Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any AAV serotype can be used, including AAV1, AAV3, AAV4, AAV5, AAV6 and AAV8, AAV 8.2, AAV9, and AAV rh10 and pseudotyped AAV such as AAV9.45, AAV2/8, AAV2/5 and AAV2/6.

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (Dunbar et al., Blood 85:3048-305 (1995); Kohn et al., Nat. Med. 1:1017-102 (1995); Malech et al., PNAS 94:22 12133-12138 (1997)). PA317/pLASN was the first therapeutic vector used in a gene therapy trial. (Blaese et al., Science 270:475-480 (1995)). Transduction efficiencies of 50% or greater have been observed for MFG-S packaged vectors. (Ellem et al., Immunol Immunother 44(1):10-20 (1997); Dranoff et al., Hum. Gene Ther. 1:111-2 (1997).

Recombinant adeno-associated virus vectors (rAAV) are promising alternative gene delivery systems based on the defective and nonpathogenic parvovirus adeno-associated type 2 virus. All vectors are derived from a plasmid that retains only the AAV 145 base pair (bp) inverted terminal repeats flanking the transgene expression cassette. Efficient gene transfer and stable transgene delivery due to integration into the genomes of the transduced cell are key features for this vector system. (Wagner et al., Lancet 351:9117 1702-3 (1998), Kearns et al., Gene Ther. 9:748-55 (1996)). Other AAV serotypes, including AAV1, AAV3, AAV4, AAV5, AAV6, AAV8, AAV8.2, AAV9, AAV9.45 and AAVrh10, and pseudotyped AAV such as AAV2/8, AAV2/5 and AAV2/6 can also be used in accordance with the present invention. In some embodiments, AAV serotypes that target cardiac, lung, brain and/or muscle are used, including but not limited to AAV serotypes that are capable of crossing the blood brain barrier are used.

Replication-deficient recombinant adenoviral vectors (Ad) can be produced at high titer and readily infect a number of different cell types. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including non-dividing, differentiated cells such as those found in liver, kidney and muscle. Conventional Ad vectors have a large carrying capacity. An example of the use of an Ad vector in a clinical trial involved polynucleotide therapy for anti-tumor immunization with intramuscular injection (Sterman et al., Hum. Gene Ther. 7:1083-9 (1998)). Additional examples of the use of adenovirus vectors for gene transfer in clinical trials include Rosenecker et al., Infection 24:1 5-10 (1996); Sterman et al., Hum. Gene Ther. 9:7 1083-1089 (1998); Welsh et al., Hum. Gene Ther. 2:205-18 (1995); Alvarez et al., Hum. Gene Ther. 5:597-613 (1997); Topf et al., Gene Ther. 5:507-513 (1998); Sterman et al., Hum. Gene Ther. 7:1083-1089 (1998).

Packaging cells are used to form virus particles that are capable of infecting a host cell. Such cells include 293 cells, which package adenovirus, and ψ2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al., Proc. Natl. Acad. Sci. USA 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual subject, typically by systemic administration (e.g., intravenous, intraperitoneal, intramuscular, subdermal, sublingual or intracranial infusion) topical application, as described below, or via pulmonary inhalation. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Vectors (e.g., retroviruses, adenoviruses, liposomes, etc.) containing nucleases and/or donor constructs can also be administered directly to an organism for transduction of cells in vivo. Alternatively, naked DNA can be administered. Administration is by any of the routes normally used for introducing a molecule into ultimate contact with blood or tissue cells including, but not limited to, injection, infusion, topical application, inhalation and electroporation. Suitable methods of administering such nucleic acids are available and well known to those of skill in the art, and, although more than one route can be used to administer a particular composition, a particular route can often provide a more immediate and more effective reaction than another route.

Vectors suitable for introduction of polynucleotides described herein include non-integrating lentivirus vectors (IDLV). See, for example, Ory et al. (1996) Proc. Natl. Acad. Sci. USA 93:11382-11388; Dull et al. (1998) J. Virol. 72:8463-8471; Zuffery et al. (1998) J. Virol. 72:9873-9880; Follenzi et al. (2000) Nature Genetics 25:217-222; U.S. Pat. No. 8,936,936.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17th ed., 1989).

It will be apparent that the nuclease-encoding sequences and donor constructs can be delivered using the same or different systems. For example, a donor polynucleotide can be carried by an AAV, while the one or more nucleases can be carried by mRNA. Furthermore, the different systems can be administered by the same or different routes (intramuscular injection, tail vein injection, other intravenous injection, intraperitoneal administration and/or intramuscular injection. Multiple vectors can be delivered simultaneously or in any sequential order.

Formulations for both ex vivo and in vivo administrations include suspensions in liquid or emulsified liquids. The active ingredients often are mixed with excipients which are pharmaceutically acceptable and compatible with the active ingredient. Suitable excipients include, for example, water, saline, dextrose, glycerol, ethanol or the like, and combinations thereof. In addition, the composition may contain minor amounts of auxiliary substances, such as, wetting or emulsifying agents, pH buffering agents, stabilizing agents or other reagents that enhance the effectiveness of the pharmaceutical composition.

The effective amount to be administered for modification of mtDNA, including for treatment and/or prevention of mitochondrial disorders, will vary from subject to subject and according to the mode of administration and site of administration. Accordingly, effective amounts are best determined by the person administering the compositions and appropriate dosages can be determined readily by one of ordinary skill in the art. In certain embodiments, after allowing sufficient time for expression (typically 2-15 days or more, for example), analysis of the serum or other tissue levels for mtDNA modification and comparison to prior to administration will determine whether the amount being administered is too low, within the right range or too high. Suitable regimes for initial and subsequent administrations are also variable, but are typified by an initial administration followed by subsequent administrations if necessary. Subsequent administrations may be administered at variable intervals, ranging from daily to annually to every several years. In certain embodiments, when using a viral vector such as AAV, the total or component dose administered may be between 1×10¹⁰ and 5×10¹⁵ vg/ml (or any value therebetween), even more preferably between 1×10¹¹ and 1×10¹⁴ vg/ml (or any value therebetween), even more preferably between 1×10¹² and 1×10¹³ vg/ml (or any value therebetween). In some embodiments, the total dose may be administered intravenously and may be between 5e12 vg/kg and 1e15 vg/kg (or any value therebetween), even more preferably between 5e13 vg/kg and 5e14 vg/kg (or any value therebetween), even more preferably between 5e13 vg/kg and 1e14 vg/kg (or any value therebetween).

### Applications

The methods and compositions disclosed herein are for providing therapies for mitochondrial diseases and disorders, for example by modifying the heteroplasmic ratio a mutant mtDNA to wild-type DNA such that the disease or disorder is treated and/or prevented. The cell may be modified in vivo or may be modified ex vivo and subsequently administered to a subject. Thus, the methods and compositions provide for the treatment and/or prevention of a mitochondrial disorder.

Non-limiting examples of mitochondrial disorders that can be treated and/or prevented using the methods and compositions described herein include: LHON (Leber Hereditary Optic Neuropathy), MM (Mitochondrial Myopathy), AD (Alzeimer's Disease), LIMM (Lethal Infantile Mitochondrial Myopathy), ADPD (Alzeimer's Disease and Parkinson's Disease), MMC (Maternal Myopathy and Cardiomyopathy), NARP (Neurogenic muscle weakness, Ataxia, and Retinitis Pigmentosa; alternate phenotype at this locus reported as Leigh Disease), FICP (Fatal Infantile Cardiomyopathy Plus, a MELAS-associated cardiomyopathy), MELAS (Mitochondrial Encephalomyopathy, Lactic Acidosis, and Stroke-like episodes), LDYT (Leber's hereditary optic neuropathy and DysTonia), MERRF (Myoclonic Epilepsy and Ragged Red Muscle Fibers), MHCM (Maternally inherited Hypertrophic CardioMyopathy), CPEO (Chronic Progressive External Ophthalmoplegia), KSS (Kearns Sayre Syndrome), DM (Diabetes Mellitus), DMDF (Diabetes Mellitus+DeaFness), CIPO (Chronic Intestinal Pseudoobstruction with myopathy and Ophthalmoplegia), DEAF (Maternally inherited DEAFness or aminoglycoside-induced DEAFness), PEM (Progressive encephalopathy), SNHL (SensoriNeural Hearing Loss), aging, encephalomyopathy, FBSN (familial bilateral striatal necrosis), PEO, and SNE (subacute necrotizing encephalopathy)

Nuclease-mediated cleavage may be used to correct mtDNA sequences associated with disease (e.g., point mutations, substitution mutations, etc.). Correction may be via degradation of the cleaved mtDNA sequences, for example in the absence of efficient DNA repair mechanisms as is typically the case in mitochondria. Specific mutant human mtDNAs that may be targeted include 1555G, 1624T, 3243G, 3460A, 3271C, 4300G, 5545T, 7445G, 7472 insertions, 8344G, 8356C 8993G, 9176G/C, 10158C, 10191C, 10197A, 11777A, 11778A, 13513A, 14459A, 14484C, 14487C, and 14709C.

By way of non-limiting example, the methods and compositions described herein can be used for treatment and/or prevention of mitochondrial disorders including but not limited to Mitochondrial myopathy; Diabetes mellitus and deafness (DAD); Leber's; Leber's hereditary optic neuropathy (LHON) which is characterized by progressive loss of central vision due to degeneration of the optic nerves and retina which affects 1 in 50,000 people in Finland; Leigh syndrome; Maternally inherited Leigh syndrome; Leigh-like syndrome; Neuropathy, ataxia, retinitis pigmentosa, and ptosis (NARP); Myoneurogenic gastrointestinal encephalopathy (MNGIE); Myoclonic Epilepsy with Ragged Red Fibers (MERRF); Mitochondrial myopathy, encephalomyopathy, lactic acidosis, stroke-like symptoms (MELAS); mtDNA depletion mitochondrial neurogastrointestinal encephalomyopathy (MNGIE), cardiomyopathies, deafness, others.

The following Examples relate to exemplary embodiments of the present disclosure in which the nuclease comprises a zinc finger nuclease (ZFN). It will be appreciated that this is for purposes of exemplification only and that other nucleases can be used, for example TALEN, TtAgo and CRISPR/Cas systems, homing endonucleases (meganucleases) with engineered DNA-binding domains and/or fusions of naturally occurring of engineered homing endonucleases (meganucleases) DNA-binding domains and heterologous cleavage domains and/or fusions of meganucleases and TALE proteins. For instance, additional nucleases may be designed to bind to a sequence comprising 9 to 12 contiguous nucleotides of the sequences disclosed herein (e.g., Table 2). In addition, the following examples relate to nucleases in which the DNA-binding domain (ZFP, TAL-effector domain, sgRNA, etc.) binds selectively to mtDNA having a 5024C>T mutation (as shown in Table 2). It will be apparent that this is for purposes of exemplification only and nucleases that bind to other mutant mtDNA sequences are contemplated, including but not limited to one or more mutations at one or more of the following locations: 1555G, 1624T, 3243G, 3460A, 3271C, 4300G, 5545T, 7445G, 7472 random insertions, 8344G, 8356C 8993G, 9176G/C, 10158C, 10191C, 10197A, 11777A, 11778A, 13513A, 14459A, 14484C, 14487C and/or 14709C.

### EXAMPLES

### Example 1: mtDNA Nucleases

Zinc finger proteins targeted to mtDNA were designed and incorporated into mRNA, plasmids, AAV or adenoviral vectors essentially as described in Gammage et al. (2014) EMBO Mol Med. 6:458-466; Gammage et al. (2016) Methods in Mol. Biol. 1351:145-162; in U.S. Pat. Nos. 6,534,261 and 9,139,628.

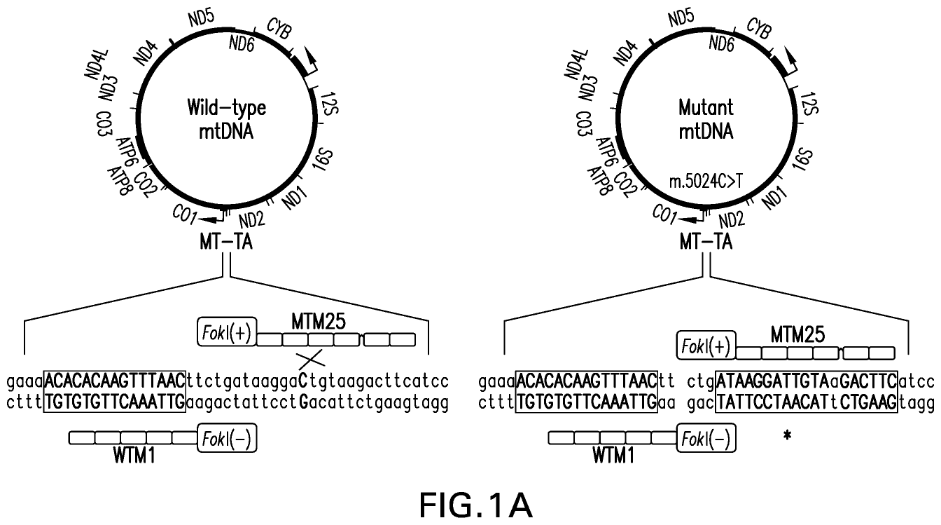

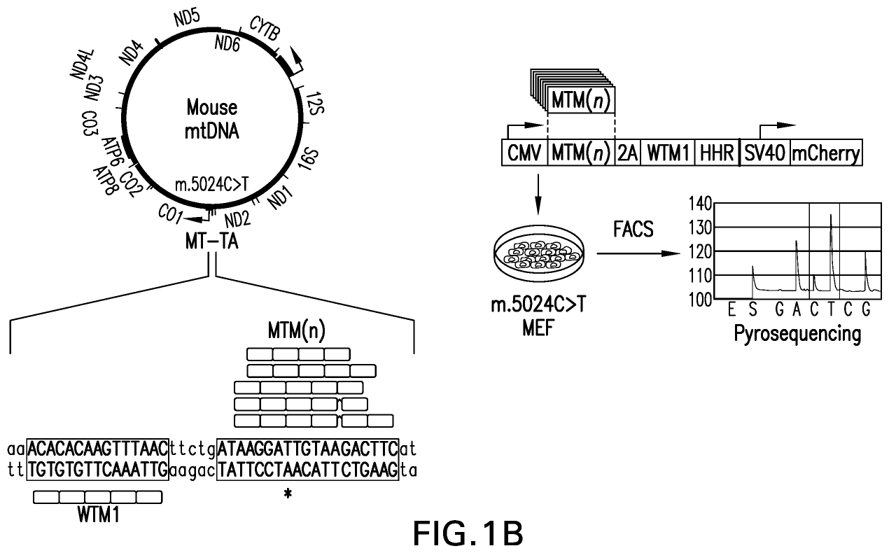

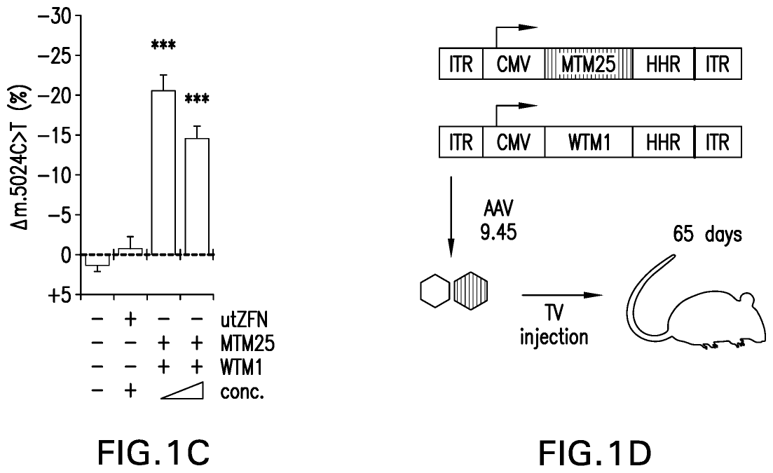

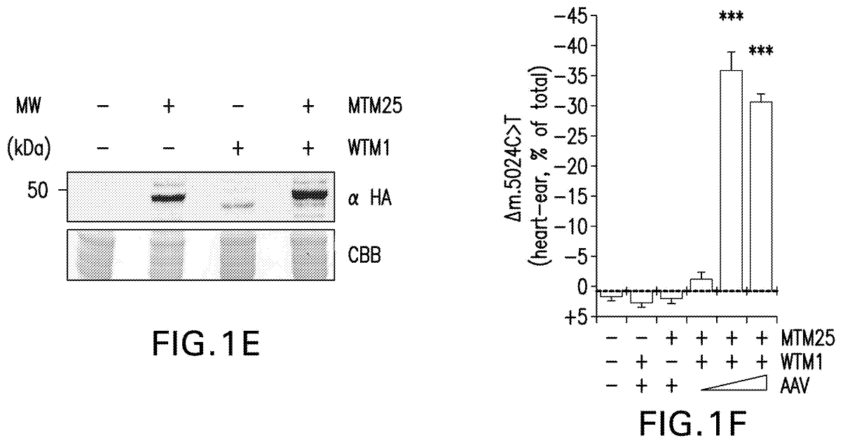

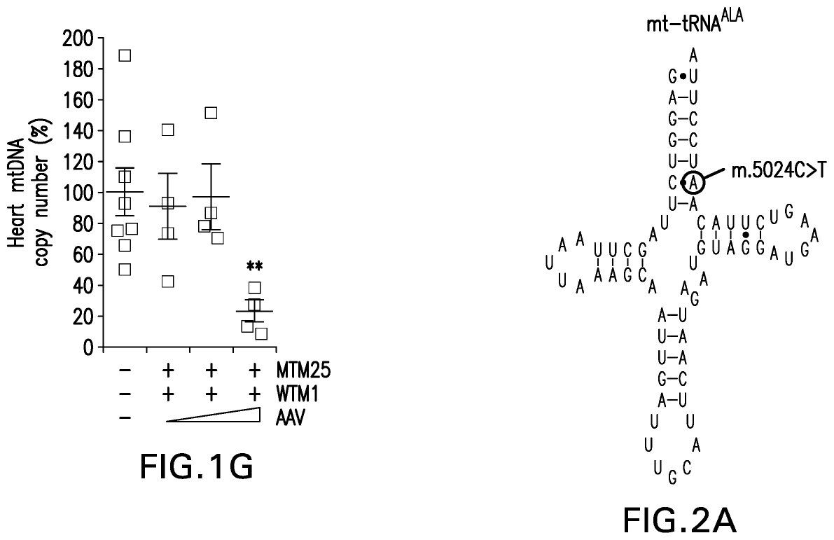

Specifically, pairs of ZFPs with single nucleotide binding specificity for mutant mt DNA (m5024C>T) were generated. See, FIG. 1. As this site in the mouse mtDNA is challenging for ZFPs, a selection of targeting strategies with varying numbers of zinc finger motifs, spacer region lengths and additional linkers were employed. Assembly of candidate ZFPs yielded a library (FIG. 1A) of 24 unique ZFPs targeting the m.5024C>T site, referred to as mutant-specific monomer (MTM), and a single partner ZFP targeting an adjacent sequence on the opposite strand, referred to as wild-type-specific monomer 1 (WTM1).

The MTM(n)_ T2A_WTM1 m.5024C>T candidate library was cloned by insertion of the MTM ZFP domains upstream of FokI(+) between 5′ EcoRI and 3′ BamHI restriction sites. This product was then PCR amplified to include a 5′ ApaI site and remove the 3′ stop codon while also incorporating a T2A sequence and 3′ XhoI site. This fragment was then cloned into pcmCherry (Addgene 62803) using ApaI/XhoI sites. The WTM1 ZFP was separately cloned upstream of FokI(−) in the pcmCherry_3k19 vector (Addgene 104499) incorporating the 3′ hammerhead ribozyme (HEIR) using 5′ EcoRI and 3′ BamHI sites, and the resulting product was PCR amplified to include 5′ XhoI and 3′ AflII sites allowing cloning downstream of MTM(n) variants.

MTM25(+) and WTM1(−) monomers were also cloned into separate pcmCherry and pTracer vectors as described previously in Gammage et al. (2016) Methods in Mol. Biol. 1351:145-162. Vector construction of mtZFNs intended for AAV production was achieved by PCR amplification of MTM25(+)_HHR and WTM1(−)_HHR transgenes, incorporating 5′ EagI and 3′ BglII sites.

These products were then cloned into rAAV2-CMV between 5′ EagI and 3′ BamHI sites. The FLAG epitope tag of WTM1(−) was replaced with a hemagluttinin (HA) tag by PCR. The resulting plasmids were used to generate recombinant AAV2/9.45-CMV-MTM25 and AAV2/9.45-CMV-WTM1 viral particles at the UNC Gene Therapy Center, Vector Core Facility (Chapel Hill, N.C.). The 3K19 hammerhead ribozyme (HEIR) sequence (Beilstein et al. (2015) ACS Synth Biol 4:526-534) was incorporated into mtZFN-AAV9.45 constructs to allow ubiquitous expression of the transgene from CMV while limiting the expression level, allowing administration of the high viral titers required to ensure effective co-transduction of cells in the targeted tissue without inducing large mtDNA copy number depletions.

Table 1 shows the recognition helices within the DNA binding domain of exemplary mtDNA ZFP DNA-binding domains and the target sites for these ZFPs (DNA target sites indicated in uppercase letters; non-contacted nucleotides indicated in lowercase). Nucleotides in the target site that are contacted by the ZFP recognition helices are indicated in uppercase letters; non-contacted nucleotides indicated in lowercase. TALENs and/or sgRNAs are also designed to the sequences shown in Table 2 (e.g., a target site comprising 9 to 20 or more (including 9, 10, 11, 12, 13, 14, 15, 16, 17, 18, 19, 20, 21, or more) nucleotides (contiguous or non-contiguous) of the target sites shown in Table 2 following methods known in the art. See, e.g., U.S. Pat. No. 8,586,526 (using canonical or non-canonical RVDs for TALENs) and U.S. Patent Publication No. 2015/0056705.

TABLE 1
mtDNA Zinc finger proteins recognition helix designs

| | | Design | | | | | |
|---|---|---|---|---|---|---|---|
| SBS # | Linker | F1 | F2 | F3 | F4 | F5 | F6 |
| WTM1 | 5, 6 | LPHHLEQ | PNASRTR | YTYSLSE | QSANRTT | HRSSLRR | N/A |
| 48960 | | (SEQ ID | (SEQ ID | (SEQ ID | (SEQ ID | (SEQ ID | |
| (Left) | | NO: 2) | NO: 3) | NO: 4) | NO: 5) | NO: 6) | |

Right ZFNs, 5-bp gap with 48960

| | | | | | | | |
|---|---|---|---|---|---|---|---|
| 48962 | 5, 6 | GNTGLNC | DRSNLTR | QSGSLTR | HKSARAA | RSDHLSA | QHGALQT |
| | | (SEQ ID | (SEQ ID | (SEQ ID | (SEQ ID | (SEQ ID | (SEQ ID |
| | | NO: 7) | NO: 8) | NO: 9) | NO: 10) | NO: 11) | NO: 12) |
| 51024 | 5, 6 | GNTGLNC | DRSNLTR | QSGSLTR | HKSARAA | RSDHLSQ | QSNGLTQ |
| | | (SEQ ID | (SEQ ID | (SEQ ID | (SEQ ID | (SEQ ID | (SEQ ID |
| | | NO: 7) | NO: 8) | NO: 9) | NO: 10) | NO: 13) | NO: 14) |
| 51025 | 5, 6 | GNTGLNC | DRSNLTR | QSGSLTR | HKSARAA | RSDHLSA | QHGSLAS |
| | | (SEQ ID | (SEQ ID | (SEQ ID | (SEQ ID | (SEQ ID | (SEQ ID |
| | | NO: 7) | NO: 8) | NO: 9) | NO: 10) | NO: 11) | NO: 15) |
| 51026 | 5, 6 | GNTGLNC | DRSNLTR | QSGSLTR | HKSARAA | RSAHLSA | QHGALQT |
| | | (SEQ ID | (SEQ ID | (SEQ ID | (SEQ ID | (SEQ ID | (SEQ ID |
| | | NO: 7) | NO: 8) | NO: 9) | NO: 10) | NO: 16) | NO: 12) |
| 51027 | 5, 6 | GNTGLNC | DRSNLTR | QSGSLTR | HKSARAA | RSAHLSA | SSSHRCQ |
| | | (SEQ ID | (SEQ ID | (SEQ ID | (SEQ ID | (SEQ ID | (SEQ ID |
| | | NO: 7) | NO: 8) | NO: 9) | NO: 10) | NO: 16) | NO: 17) |
| 51028 | 5, 6 | GNTGLNC | DRSNLTR | QSGSLTR | HKSARAA | RSAHLSA | QRVALQA |
| | | (SEQ ID | (SEQ ID | (SEQ ID | (SEQ ID | (SEQ ID | (SEQ ID |
| | | NO: 7) | NO: 8) | NO: 9) | NO: 10) | NO: 16) | NO: 18) |
| 51029 | 5, 6 | GNTGLNC | DRSNLTR | QSGALAR | HKSARAA | RSDHLSA | QHGALQT |
| | | (SEQ ID | (SEQ ID | (SEQ ID | (SEQ ID | (SEQ ID | (SEQ ID |
| | | NO: 7) | NO: 8) | NO: 19) | NO: 10) | NO: 11) | NO: 12) |
| 51030 | 5, 6 | GNTGLNC | DRSNLTR | QSGALAR | HKSARAA | RSDHLSA | WYTARYQ |
| | | (SEQ ID | (SEQ ID | (SEQ ID | (SEQ ID | (SEQ ID | (SEQ ID |
| | | NO: 7) | NO: 8) | NO: 19) | NO: 10) | NO: 11) | NO: 21) |
| 51032 | 5, 6 | GNTGLNC | DRSNLTR | QSGALAR | HKSARAA | RSDHLSA | QHGSLAS |
| | | (SEQ ID | (SEQ ID | (SEQ ID | (SEQ ID | (SEQ ID | (SEQ ID |
| | | NO: 7) | NO: 8) | NO: 19) | NO: 10) | NO: 11) | NO: 15) |
| 51033 | 5, 6 | GNTGLNC | DRSNLTR | QSGALAR | HKSARAA | RSAHLSA | QHGALQT |
| | | (SEQ ID | (SEQ ID | (SEQ ID | (SEQ ID | (SEQ ID | (SEQ ID |
| | | NO: 7) | NO: 8) | NO: 19) | NO: 10) | NO: 16) | NO: 12) |
| 51036 | 5, 6 | GNTGLNC | DRSNLTR | QSGALAR | YRWLRNS | RSDHLSA | QHGALQT |
| | | (SEQ ID | (SEQ ID | (SEQ ID | (SEQ ID | (SEQ ID | (SEQ ID |
| | | NO: 7) | NO: 8) | NO: 19) | NO: 20) | NO: 11) | NO: 12) |
| 51037 | 5, 6 | GNTGLNC | DRSNLTR | QSGALAR | YRWLRNS | RSDHLSA | WYTARYQ |
| | | (SEQ ID | (SEQ ID | (SEQ ID | (SEQ ID | (SEQ ID | (SEQ ID |
| | | NO: 7) | NO: 8) | NO: 19) | NO: 20) | NO: 11) | NO: 21) |
| 51039 | 5, 6 | GNTGLNC | DRSNLTR | QSGALAR | YRWLRNS | RSDHLSA | QHGSLAS |
| | | (SEQ ID | (SEQ ID | (SEQ ID | (SEQ ID | (SEQ ID | (SEQ ID |
| | | NO: 7) | NO: 8) | NO: 19) | NO: 20) | NO: 11) | NO: 15) |
| 51042 | 5, 6 | GNTGLNC | DRSNLTR | QSGALAR | YRWLRNS | RSAHLSA | QRVALQA |
| | | (SEQ ID | (SEQ ID | (SEQ ID | (SEQ ID | (SEQ ID | (SEQ ID |
| | | NO: 7) | NO: 8) | NO: 19) | NO: 20) | NO: 16) | NO: 18) |
| 51043 | 5, 6 | DRSNLTR | QSGSLTR | HKSARAA | RSDHLSA | QHGALQT | N/A |
| | | (SEQ ID | (SEQ ID | (SEQ ID | (SEQ ID | (SEQ ID | |
| | | NO: 8) | NO: 9) | NO: 10) | NO: 11) | NO: 12) | |
| 51045 | 5, 6 | QRTHLTQ | QSGSLTR | HKSARAA | RSDHLSA | QHGALQT | N/A |
| | | (SEQ ID | (SEQ ID | (SEQ ID | (SEQ ID | (SEQ ID | |
| | | NO: 22) | NO: 9) | NO: 10) | NO: 11) | NO: 12) | |

Right ZFNs, 6 bp gap with 48960

| | | | | | | | |
|---|---|---|---|---|---|---|---|
| 48965 | 5, 6 | DRSNLSR | QQANRKK | RPYTLRL | QSGHLAR | QSSNRQK | N/A |
| | | (SEQ ID | (SEQ ID | (SEQ ID | (SEQ ID | (SEQ ID | |
| | | NO: 23) | NO: 24) | NO: 25) | NO: 26) | NO: 27) | |
| 48966 | 5, 6 | DRSNLSR | QQANRKK | RSFSLQV | QSGHLAR | QSSNRQK | N/A |
| | | (SEQ ID | (SEQ ID | (SEQ ID | (SEQ ID | (SEQ ID | |
| | | NO: 23) | NO: 24) | NO: 28) | NO: 26) | NO: 27) | |
| 51048 | 5, 6 | DRSNLSR | QQANRKK | RTYSLAV | QSGHLAR | QSSNRQK | N/A |
| | | (SEQ ID | (SEQ ID | (SEQ ID | (SEQ ID | (SEQ ID | |
| | | NO: 23) | NO: 24) | NO: 29) | NO: 26) | NO: 27) | |
| 51049 | 5, 6 | DRSNLSR | QQANRKK | RNFSLTM | QSGHLAR | QSSNRQK | N/A |
| | | (SEQ ID | (SEQ ID | (SEQ ID | (SEQ ID | (SEQ ID | |
| | | NO: 23) | NO: 24) | NO: 30) | NO: 26) | NO: 27) | |
| 51050 | 5, 6 | DRSNLSR | QQANRKK | QWYGRSN | QSGHLAR | QSSNRQK | |
| | | (SEQ ID | (SEQ ID | (SEQ ID | (SEQ ID | (SEQ ID | |
| | | NO: 23) | NO: 24) | NO: 31) | NO: 26) | NO: 27) | |
| 51052 | 5, 6 | QSANRTK | RSFSLQV | QSGHLAR | QSSNRQK | N/A | N/A |
| | | (SEQ ID | (SEQ ID | (SEQ ID | (SEQ ID | | |
| | | NO: 32) | NO: 28) | NO: 26) | NO: 27) | | |
| 51055 | 5, 6 | DRSNLTR | QSANRTK | RSFSLQV | QSGHLAR | QSSNRQK | N/A |
| | | (SEQ ID | (SEQ ID | (SEQ ID | (SEQ ID | (SEQ ID | |
| | | NO: 8) | NO: 32) | NO: 28) | NO: 26) | NO: 27) | |
| 51056 | 5, 6 | DRSNLTR | QSANRTK | RSFTLMQ | QSGHLAR | QSSNRQK | |
| | | (SEQ ID | (SEQ ID | (SEQ ID | (SEQ ID | (SEQ ID | |
| | | NO: 8) | NO: 32) | NO: 48) | NO: 26) | NO: 27) | |

TABLE 2
Target Sites of zinc finger proteins

| MTM # | SBS # | Target site |
|---|---|---|
| WTM1 | 48960 | aaGTTAAACTTGTGTGTtttcttagggc (SEQ ID NO: 33) |
| MTM62 | 48962 | tgATAAGGATTGTAaGACTTCatcctac (SEQ ID NO: 34) |
| MTM24 | 51024 | tgATAAGGATTGTAaGACTTCatcctac (SEQ ID NO: 34) |
| MTM25 | 51025 | tgATAAGGATTGTAaGACTTCatcctac (SEQ ID NO: 34) |
| MTM26 | 51026 | tgATAAGGATTGTAaGACTTCatcctac (SEQ ID NO: 34) |
| MTM27 | 51027 | tgATAAGGATTGTAaGACTTCatcctac (SEQ ID NO: 34) |
| MTM28 | 51028 | tgATAAGGATTGTAaGACTTCatcctac (SEQ ID NO: 34) |
| MTM29 | 51029 | tgATAAGGATTGTAaGACTTCatcctac (SEQ ID NO: 34) |
| MTM30 | 51030 | tgATAAGGATTGTAaGACTTCatcctac (SEQ ID NO: 34) |
| MTM32 | 51032 | tgATAAGGATTGTAaGACTTCatcctac (SEQ ID NO: 34) |
| MTM33 | 51033 | tgATAAGGATTGTAaGACTTCatcctac (SEQ ID NO: 34) |
| MTM36 | 51036 | tgATAAGGATTGTAaGACTTCatcctac (SEQ ID NO: 34) |
| MTM37 | 51037 | tgATAAGGATTGTAaGACTTCatcctac (SEQ ID NO: 34) |
| MTM39 | 51039 | tgATAAGGATTGTAaGACTTCatcctac (SEQ ID NO: 34) |
| MTM42 | 51042 | tgATAAGGATTGTAaGACTTCatcctac (SEQ ID NO: 34) |
| MTM43 | 51043 | tgATAAGGATTGTAaGACttcatcctac (SEQ ID NO: 34) |
| MTM45 | 51045 | tgATAAGGATTGTAAGActtcatcctac (SEQ ID NO: 34) |
| MTM65 | 48965 | gaTAAGGATTGTAAGACttcatcctaca (SEQ ID NO: 35) |
| MTM66 | 48966 | gaTAAGGATTGTAAGACttcatcctaca (SEQ ID NO: 35) |
| MTM48 | 51048 | gaTAAGGATTGTAAGACttcatcctaca (SEQ ID NO: 35) |
| MTM49 | 51049 | gaTAAGGATTGTAAGACttcatcctaca (SEQ ID NO: 35) |
| MTM50 | 51050 | gaTAAGGATTGTAAGACttcatcctaca (SEQ ID NO: 35) |
| MTM52 | 51052 | gaTAAGGATTGTAAgacttcatcctaca (SEQ ID NO: 35) |
| MTM55 | 51055 | gaTAAGGATTGTAAGACttcatcctaca (SEQ ID NO: 35) |
| MTM56 | 51056 | gaTAAGGATTGTAAGACttcatcctaca (SEQ ID NO: 35) |

All ZFN pairwise combinations were tested for cleavage activity. Wild-type and m.5024C>T mouse embryonic fibroblast (MEF) cell lines were cultured in Dulbecco's Modified Eagle's Medium (DMEM) containing 2 mM L-glutamine, 110 mg/L sodium pyruvate (Life Technologies) and 10% FCS (PAA Laboratories). Cells were transfected by electroporation using Nucleofector II apparatus (Lonza) using a MEF1 kit and T20 program. Fluorescence activated cell sorting (FACS) was performed as described in Gammage et al. (2016) Methods Mol Biol 1352:145-162. Control of mtZFN expression was achieved through titration of tetracycline into culture media, controlling the rate of HHR autocatalysis as described previously in Gammage et al. (2016) Nucleic Acids Res 44:7804, which also describes how total cellular protein extraction were performed. Detection of proteins by western blotting was achieved by resolving 20-100m of extracted protein on SDS-PAGE 4-12% bis-tris Bolt gels. These were transferred to nitrocellulose using an iBlot 2 transfer cell (Life Technologies). Antibodies used for western blotting in this work: rat anti-HA (Roche, 1:500), goat anti-rat HRP (Santa Cruz, 1:1000). Gels were stained for loading using Coomassie Brilliant Blue (Life Technologies). All pairs were found to be active.

Constructs were also subjected to several rounds of screening in mouse embryonic fibroblasts (MEFs) bearing ˜65% m.5024C>T to assess heteroplasmy shifting activity. As shown in FIG. 1, these screens identified consistent, specific activity of pairing MTM25/WTM1, which produced a shift of ˜20%, from 65% to 45% m.5024C>T in the MEF cell line as determined by pyrosequencing. Briefly, assessment of m.5024C>T mtDNA heteroplasmy was carried out by pyrosequencing. PCR reactions for pyrosequencing were prepared using KOD DNA polymerase (Takara) for 40 cycles using 100 ng template DNA with the following primers:

m.4,962-4,986 Forward
(SEQ ID NO: 36)
5′ ATACTAGTCCGCGAGCCTTCAAAG 3′
m.5,360-m.5,383 Reverse
(SEQ ID NO: 37)
5′ [Btn] GAGGGTTCCGATATCTTTGTGATT 3′
m.5003-m.5022 Sequencing primer
(SEQ ID NO: 38)
5′ AAGTTTAACTTCTGATAAGG 3′

In addition, mitochondrial localization was confirmed by immunofluorescence in fixed MEF cells as described in Minczuk et al. (2010) Methods Mol Biol 649:257-270. MTM25 and WTM1 were localized exclusively in mitochondria and this pair was selected for further in vivo experiments.

It will be apparent that these designs may include any linker between any of the finger modules and/or between the ZFP and the cleavage domain, including but not limited to canonical or non-canonical linkers (between fingers) and/or linkers between the ZFP and cleavage domain as described in U.S. Pat. No. 9,394,531. See, also, U.S. Pat. No. 8,772,453 and U.S. Patent Publication No. 2015/0064789.

Furthermore, nucleases other than ZFNs, including CRISPR/Cas nucleases, TALENs etc., can be designed to target sites of 9-18 or more nucleotides as shown above. Any of the nucleases (ZFNs, CRISPR/Cas systems and TALENs) can include engineered cleavage domains, for example heterodimers disclosed in U.S. Pat. No. 8,623,618 (e.g., ELD and KKR engineered cleavage domains) and/or cleavage domains with more or more mutations in positions 416, 422, 447, 448, and/or 525 as described in U.S. Patent Publication No. 20180087072. These mutants were used in conjunction with the exemplary ZFP DNA-binding domains described herein.

### Example 2: In Vivo Nuclease Activity

Nuclease activity was also tested in vivo in mice. The C57BL/6j-tRNA^{ALA} mice used in this study were housed from one to four per cage in a temperature controlled (21° C.) room with a 12 h light-dark cycle and 60% relative humidity.

MTM25 and WTM1 mtZFN monomers were encoded in separate viral genomes and encapsidated within the cardiac-tropic, engineered AAV9.45 serotype (FIG. 1D). See, Pulicheria et al. (2011) Mol Ther 19:1070-1078. Detection of proteins by western blotting was achieved by resolving 20-100 μg of extracted protein on SDS-PAGE 4-12% bis-tris Bolt gels. These were transferred to nitrocellulose using an iBlot 2 transfer cell (Life Technologies). Antibodies used for western blotting in this work: rat anti-HA (Roche, 11867431001, 1:500), goat anti-rat HRP (Santa Cruz, SC2065, 1:1000). Gels were stained for loading using Coomassie Brilliant Blue (Life Technologies).

As shown in FIG. 1E, following systemic (tail-vein) administration of 5×10¹² viral genomes (vg) per monomer per mouse, robust expression of MTM25 and WTM1 in total mouse heart tissue was detected by western blotting.

Further in vivo experiments were carried out as follows. Male mice between 2 to 8 months of age harboring 44%-81% m.5024C>T heteroplasmy (20 Vehicle, 7 Single Monomer, 4 per mtZFN-AAV9.45 dosage) were treated in the groups as shown below:

| | | Ear | | | mDNA | Mt- | |
|---|---|---|---|---|---|---|---|
| | | genotype | Heart | Δm.5024C > T | copy | RNA^{ALA} | |
| Subject | Condition | [E] | genotype [H] | [H-E] | no. | analysis | LC/MS |
| 1 | Veh. | 54 | 50 | −4 | N | N | N |
| 2 | Veh. | 50 | 55 | 5 | N | N | N |
| 3 | Veh. | 48 | 55 | 7 | N | N | N |
| 4 | Veh. | 51 | 50 | −1 | N | N | N |
| 5 | Veh. | 53 | 53 | 0 | N | N | N |
| 6 | Veh. | 53 | 54 | 1 | N | N | N |
| 7 | Veh. | 56 | 64 | 8 | N | N | N |
| 8 | Veh. | 64 | 58 | −6 | N | N | N |
| 9 | Veh. | 70 | 74 | 4 | N | N | N |
| 10 | Veh. | 76 | 78 | 2 | N | N | N |
| 11 | Veh. | 61 | 60 | −1 | N | N | N |
| 12 | Veh. | 66 | 56 | −10 | N | N | N |
| 13 | Veh. | 73 | 75 | 2 | Y | Y | N |
| 14 | Veh. | 72 | 73 | 1 | Y | Y | N |
| 15 | Veh. | 75 | 76 | 1 | Y | Y | N |
| 16 | Veh. | 74 | 76 | 2 | Y | Y | N |
| 17 | Veh. | 70 | 75 | 5 | Y | Y | N |
| 18 | Veh. | 67 | 72 | 5 | Y | Y | Y |
| 19 | Veh. | 71 | 71 | 0 | Y | Y | Y |
| 20 | Veh. | 68 | 69 | 1 | Y | Y | Y |
| 21 | MTM25 5*10e12vg | 49 | 54 | 5 | N | N | N |
| 22 | MTM25 5*10e12vg | 68 | 71 | 3 | N | N | N |
| 23 | MTM25 5*10e12vg | 53 | 54 | 1 | N | N | N |
| 24 | MTM25 5*10e12vg | 67 | 67 | 0 | N | N | N |
| 25 | WTM1 5*10e12vg | 50 | 54 | 4 | N | N | N |
| 26 | WTM1 5*10e12vg | 56 | 51 | −5 | N | N | N |
| 27 | WTM1 5*10e12vg | 44 | 49 | 5 | N | N | N |
| 28 | mtZFN 1*10e13vg | 68 | 37 | −31 | Y | Y | N |
| 29 | mtZFN 1*10e13v5 | 75 | 48 | −27 | Y | Y | N |
| 30 | mtZFN 1*10e13vg | 70 | 37 | −33 | Y | Y | N |
| 31 | mtZFN 1*10e13vg | 72 | 36 | −36 | Y | Y | N |
| 32 | mtZFN 5*10e12vg | 81 | 45 | −36 | Y | Y | Y |
| 33 | mtZFN 5*10e12vg | 74 | 37 | −37 | Y | Y | Y |
| 34 | mtZFN 5*10e12vg | 68 | 40 | −28 | Y | Y | Y |
| 35 | mtZFN 5*10e12vg | 68 | 25 | −43 | Y | Y | Y |
| 36 | mtZFN 1*10e12vg | 80 | 75 | −5 | Y | Y | N |
| 37 | mtZFN 1*10e12vg | 69 | 66 | −3 | Y | Y | N |
| 38 | mtZFN 1*10e12vg | 73 | 72 | −1 | Y | Y | N |
| 39 | mtZFN 1*10e12vg | 68 | 69 | 1 | Y | Y | N |

Treatments of vehicle (1×PBS, 350 mM NaCl, 5% w/v D-sorbitol) and AAVs were administered systemically by tail vein injection.

For mouse heart tissue, 50 mg was homogenized in RIPA buffer (150 mM NaCl, 50 mM Tris pH 8, 1% (v/v) Triton X-100, 0.5% (v/v) deoxycholate, 0.1% (v/v) SDS) using a gentleMACS dissociator (Miltenyi). The resulting homogenate was centrifuged at 10,000×g at 4C for 10 minutes, supernatant was then recovered and centrifuged at 10,000×g at 4C for 10 minutes. Concentration of both cellular and tissue protein extracts was determined by BCA assay (Pierce).

Assessment of mtDNA heteroplasmy by pyrosequencing was performed and expressed as the change (A) between ear punch genotype determined at two weeks of age (prior to experimental intervention) and post-mortem heart genotype. Briefly, mitochondrial DNA copy number of mouse heart samples was determined by qPCR using PowerUp SYBR Green Master Mix according to the manufacturer's protocol (Applied Biosystems). Samples were analysed using a 7900HT Fast Real-Time PCR System (Thermo Fisher). The following primers were used:

MT-COI Forward
(SEQ ID NO: 39)
5′ TGCTAGCCGCAGGCATTACT 3′
MT-COI Reverse
(SEQ ID NO: 40)
5′ CGGGATCAAAGAAAGTTGTGTTT 3′
RNaseP Forward
(SEQ ID NO: 41)
5′ GCCTACACTGGAGTCCGTGCTACT 3′
RNaseP Reverse
(SEQ ID NO: 42)
5′ CTGACCACACACGAGCTGGTAGAA 3′

All primers for pyrosequencing and qPCR were designed using NCBI reference sequences GRCm38.p6 and NC_005089.1 for the C57BL/6j mouse nuclear and mitochondrial genomes respectively.

As shown in FIGS. 1F and 1G, injected animals at 65 days post-injection revealed specific elimination of the m.5024C>T mutant mtDNA in mtZFN-treated mice, but not in vehicle- or single monomer-injected controls. The extent to which heteroplasmy was altered by mtZFN treatment followed a biphasic AAV dose-dependent trend, with the intermediate dose (5×10¹² vg) being the most efficient in eliminating m.5024C>T mutant mtDNA. The lowest (1×10¹² vg) dose did not result in heteroplasmy shifts, likely due to insufficient concentration of mtZFNs and/or mosaic transduction of the targeted tissue by AAV. The highest dose (1×10¹³ vg) exhibited diminished heteroplasmy shifting activity compared with the intermediate dose (5×10¹² vg), likely due to off-target effects resulting in partial mtDNA copy number depletions, which are not observed when lower doses are administered (FIG. 1G). The latter result is consistent with our past observations, underscoring the importance of fine-tuning mtZFN levels in mitochondria for efficient mtDNA heteroplasmy modification.

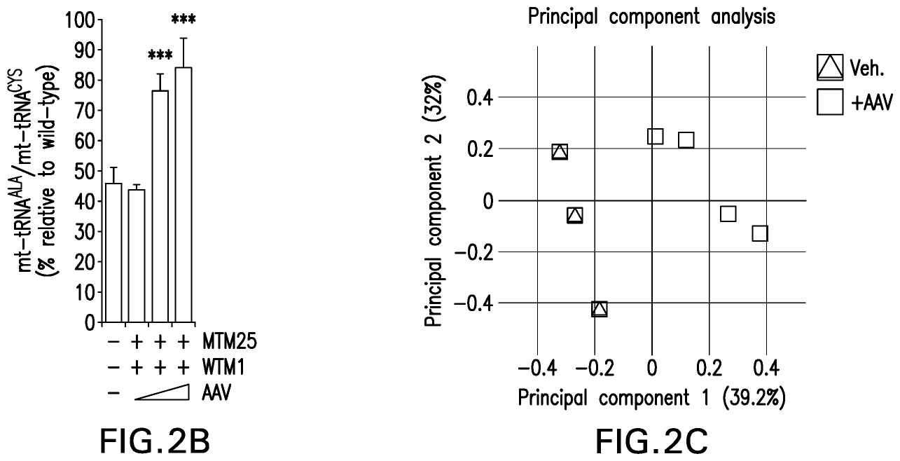

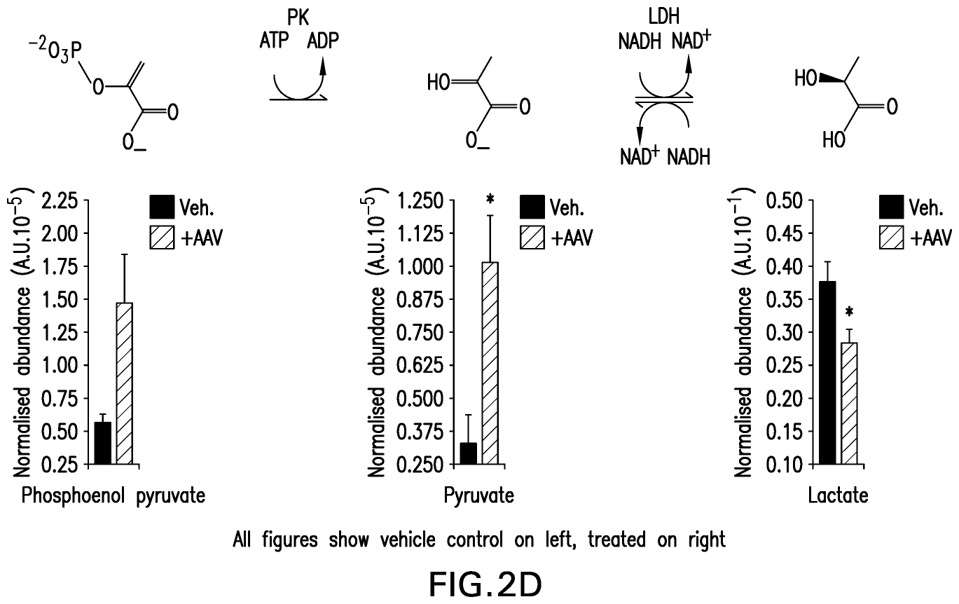

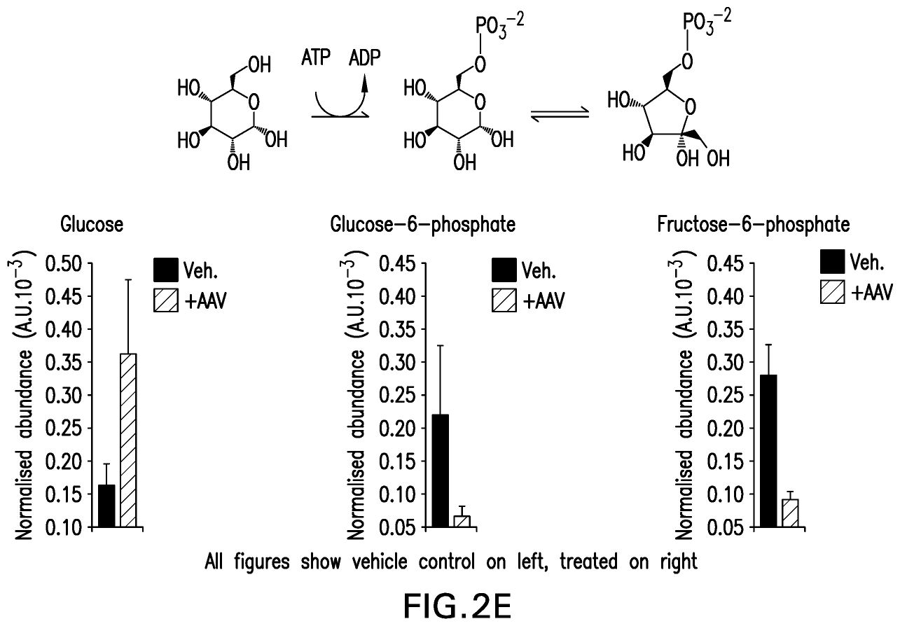

Having defined conditions within which a robust shift of m.5024C>T heteroplasmy is achieved in vivo, we next addressed disease-relevant phenotype correction in an animal model of mitochondrial disease (see, Kauppila et al. (2016) Cell Rep 16:2980-2990. A common feature of mt-tRNA mutations in mitochondrial diseases, recapitulated in the tRNAALA mouse model is the instability of mt-tRNA molecules in proportion with mutant load. See, FIG. 2A; Yarham et al. (2010) Wiley Inderdiscip Rev RNA 1:304-324.

To assess the effects of mtZFN treatment on the stability of mt-tRNAALA in the hearts of animals treated with mtZFNs across the dosage range, northern blotting was performed essentially as described in Pearce et al. (2017) Elife 6, doi:10-7554/eLife.27596. Briefly, total RNA was extracted from 25 mg of mouse heart tissue using Trizol (Ambion) by homogenization using a gentleMACS dissociator (Miltenyi). In particular, 5 ug of total RNA was resolved on a 10% polyacylamide gel containing 8 M urea. Gels were dry blotted onto a positively charged nylon membrane (Hybond-N+), with the resulting membrane cross-linked by exposure to 254 mu UV light, 120 mJ/cm². For tRNA probes, cross-linked membranes were hybridized with radioactively labelled RNA probes T7 transcribed from PCR fragments corresponding to appropriate regions of mouse mtDNA. 5S rRNA was probed with a complementary α[32P]-end labelled DNA oligo. Membranes were exposed to a storage phosphor screen and scanned using a Typhoon phosphor imaging system (GE Healthcare). The signals were quantified using Fiji software. Oligo sequences were as follows:

MT-TA Forward
(SEQ ID NO: 43)
5′ TAATACGACTCACTATAGGGAGACTAAGGACTGTAAGACTTCAT
C 3′
MT-TA Reverse
(SEQ ID NO: 44)
5′ GAGGTCTTAGCTTAATTAAAG 3′
MT-TC Forward
(SEQ ID NO: 45)
5′ TAATACGACTCACTATAGGGAGACAAGTCTTAGTAGAGATTTCT
C 3′
MT-TC Reverse
(SEQ ID NO: 46)
5′ GGTCTTAAGGTGATATTCATG 3
5S rRNA oligo:
(SEQ ID NO: 47)
5′ AAGCCTACAGCACCCGGTATTCCCAGGCGGTCTCCCATCCAAGT
ACTAACCA 3′

All primers for northern blotting were designed using NCBI reference sequences GRCm38.p6 and NC_005089.1 for the C57BL/6j mouse nuclear and mitochondrial genomes respectively.

As shown in FIG. 2B, there was significant increase in mt-tRNAALA steady-state levels that were proportional to heteroplasmy shifts detected in these mice (FIG. 1F). Depletions of mtDNA copy number associated with administration of high viral doses (FIG. 1G), did not appear to impact recovery of mt-tRNAALA steady-state levels following heteroplasmy shift, which is consistent previously published data that even severe mtDNA depletion does not manifest in proportional changes of mitochondrial RNA steady-state levels. See, Jazayeri et al. (2003) J. Biol. Chem 278:9823-9830.

Further experiments were performed to assess the physiological effects of the mt-tRNAALA molecular phenotype rescue. In particular, the steady-state metabolite abundance in cardiac tissue from mice treated with an intermediate viral titer (5×10¹² vg) was assessed. Briefly, snap-frozen tissue specimens were cut and weighed into Precellys tubes prefilled with ceramic beads (Stretton Scientific Ltd., Derbyshire, UK). An exact volume of extraction solution (30% acetonitrile, 50% methanol and 20% water) was added to obtain 40 mg specimen per mL of extraction solution. Tissue samples were lysed using a Precellys 24 homogenizer (Stretton Scientific Ltd., Derbyshire, UK). The suspension was mixed and incubated for 15 minutes at 4° C. in a Thermomixer (Eppendorf, Germany), followed by centrifugation (16,000 g, 15 min at 4° C.). The supernatant was collected and transferred into autosampler glass vials, which were stored at −80° C. until further analysis. Samples were randomized in order to avoid bias due to machine drift and processed blindly. LC-MS analysis was performed using a QExactive Orbitrap mass spectrometer coupled to a Dionex U3000 UHPLC system (Thermo). The liquid chromatography system was fitted with a Sequant ZIC-pHILIC column (150 mm×2.1 mm) and guard column (20 mm×2.1 mm) from Merck Millipore (Germany) and temperature maintained at 40° C. The mobile phase was composed of 20 mM ammonium carbonate and 0.1% ammonium hydroxide in water (solvent A), and acetonitrile (solvent B). The flow rate was set at 200 μL/min with the gradient as described previously in Mackay et al. (2015) Methods Enzymol 561:171-196. The mass spectrometer was operated in full MS and polarity switching mode. The acquired spectra were analyzed using XCalibur Qual Browser and XCalibur Quan Browser software (Thermo Scientific).

As shown in FIGS. 2C through 2E, this analysis revealed an altered metabolic signature in mtZFN treated mice (FIG. 2C), demonstrating elevated phosphoenol pyruvate and pyruvate levels, coupled to lower lactate levels as compared with controls (FIG. 2D). Additionally, treated animals exhibited higher glucose levels, but lower glucose-6-phosphate and fructose-6-phosphate levels (FIG. 2E).

Thus, recovery of mitochondrial function upon m.5024C>T heteroplasmy shift using nucleases was achieved.

In sum, the data demonstrates that nucleases targeting mutant mitochondrial DNA sequences can be used in vitro an in vivo to manipulate heteroplasmic mutations in mouse mtDNA, leading to molecular and physiological rescue of disease phenotypes in heart tissue.

All patents, patent applications and publications mentioned herein are hereby incorporated by reference in their entirety.

Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing descriptions and examples should not be construed as limiting.

1. A method of reducing or eliminating mutant mitochondrial DNA (mtDNA) in a subject in need thereof, the method comprising administering to the subject one or more polynucleotides encoding first and second zinc finger nucleases (ZFNs), wherein the first ZFN comprises a cleavage domain and a zinc finger protein (ZFP) that binds to a target site in wild-type mtDNA and the second ZFN comprises a cleavage domain and a ZFP that binds to a target site in mutant mtDNA such that mutant mtDNA in the subject is reduced or eliminated.

2. The method of claim 1, wherein the first ZFN is the left ZFN and the second ZFN is the right ZFN.

3. The method of claim 1, wherein the first and second ZFNs are encoded by different polynucleotides.

4. The method of claim 1, wherein the polynucleotides are carried by one or more AAV vectors.

5. The method of claim 1, wherein the subject is a human subject.

6. The method of claim 1, wherein the mtDNA is in the heart, brain, lung and/or muscle of the subject.

7. The method of claim 1, wherein the mutant mtDNA comprises the following mutation: m.5024C>T, 1555G, 1624T, 3243G, 3460A, 3271C, 4300G, 5545T, 7445G, 7472 random insertions, 8344G, 8356C 8993G, 9176G/C, 10158C, 10191C, 10197A, 11777A, 11778A, 13513A, 14459A, 14484C, 14487C and/or 14709C.

8. The method of claim 1, wherein the mutant mtDNA comprises the 5024C>T mutation and the left ZFP binds to a target site within SEQ ID NO:33 and the right ZFP binds to a target site within SEQ ID NO:34.

9. The method of claim 8, wherein the left ZFN comprises a ZFP designated WTM1/48960 and the right ZFN comprises a ZFP designated MTM62/48962, MTM24/51024, MTM25/51025, MTM26/51026, MTM27/51027, MTM28/51028, MTM29/51029, MTM30/51030, MTM32/51032, MTM33/51033, MTM36/51036, MTM37/51037, MTM39/51039, MTM42/51042, MTM43/51043 or MTM45/51045.

10. The method of claim 1, wherein reducing or eliminating mutant mtDNA treats a mitochondrial disease in the subject.

11. A zinc finger nuclease comprising left and right zinc finger nucleases (ZFNs), wherein the left ZFN comprises a cleavage domain and zinc finger protein (ZFP) that binds to a target site in wild-type mitochondrial DNA within SEQ ID NO:33 and the right ZFN comprises a cleavage domain and a ZFP that binds to a target site in mutant mitochondrial DNA within SEQ ID NO:34 or SEQ ID NO:35.

12. One or more polynucleotides the nuclease according to claim 11.

13. A cell comprising the zinc finger nuclease of claim 11.

14. The cell of claim 13, wherein mutant mtDNA at position 5024 in the cell is reduced or eliminated.

15. A cell or cell line produced or descended from the cell of claim 14.

16. A pharmaceutical composition comprising the zinc finger nucleases according to claim 11.

17. A kit comprising the one or more polynucleotides of claim 12.

18. One or more AAV vectors comprising the one or more polynucleotides of claim 12.

19. The cell of claim 13, wherein the cell is cardiac, brain, lung and/or muscle cell.

20. A pharmaceutical composition comprising the one or more polynucleotides of claim 12.
